(19) United States
(12) Patent Application Publication
(10) Pub. No.: US 2026/0218181 A1
(43) Pub. Date: Jul. 30, 2026

(54) SPLICE-SWITCHING OLIGONUCLEOTIDES FOR TREATING SYNGAP1-ASSOCIATED DISORDERS

(71) Applicant: THE UNIVERSITY OF CHICAGO, Chicago, IL (US)

(72) Inventors: Xiaochang ZHANG, Chicago, IL (US); Runwei YANG, Chicago, IL (US)

(73) Assignee: THE UNIVERSITY OF CHICAGO, Chicago, IL (US)

(21) Appl. No.: 19/142,746

(22) PCT Filed: Dec. 28, 2023
(86) PCT No.: PCT/US2023/086263

§ 371 Date: Jun. 24, 2025

Related U.S. Application Data
(60) Provisional application No. 63/435,744, filed on Dec. 28, 2022.
(60) Provisional application No. 63/442,388, filed on Jan. 31, 2023.

(51) Int. Cl. C12N 15/113; A61K 31/711; A61K 31/7125; A61P 25/28

(52) U.S. Cl. CPC C12N 15/113; A61K 31/711; A61K 31/7125; A61P 25/28; C12N 2310/11; C12N 2310/315; C12N 2310/321; C12N 2320/33

(57) ABSTRACT

Aspects of the disclosure relate to the discovery that antisense oligonucleotides (ASOs) that bind to a single regions of a SYNGAP1 pre-mRNA to block alternative splicing lead to an efficient increase of protein produced from matured SYNGAP1 pre-mRNA. This increase can alleviate issues related to loss-of-function mutations in the SYNGAP1 gene, including those that lead to haploinsufficiencies. Alleviating such issues can be useful to treat neurological disorders that result from such mutations.

This application claims priority of U.S. Provisional Application Nos. 63/435,744, filed Dec. 28, 2022, and 63/442,388, filed Jan. 31, 2023, both of which are hereby incorporated by reference in their entirety.

This invention was made with government support under KO1MH109747 and R01MH130594 awarded by the National Institute of Health. The government has certain rights in the invention.

The instant application contains a Sequence Listing which has been submitted in XML format and is hereby incorporated by reference in its entirety. Said XML copy, created on Dec. 27, 2023, is named ARCD.P0787WO—Seq Listing.xml and is 11,471 bytes in size.

### BACKGROUND

### I. Field of the Invention

This disclosure relates to the fields of neurology, molecular biology, and medicine.

### II. Background

Synaptic transmission and plasticity are fundamental to neuronal functions, and alterations of synaptic protein expression are direct causes of autism, intellectual disability (ID), and epilepsy (Sudhof, 2018; Zoghbi and Bear, 2012). De novo loss-of-function (LoF) mutations in SYNGAP1, encoding the synaptic Ras GTPase activating protein (Chen et al., 1998; Kim et al., 1998), are among the most prevalent causes of intellectual disabilities (ID) and autism spectrum disorders (Hamdan et al., 2009; Satterstrom et al., 2020; Vlaskamp et al., 2019). Previous human and mouse studies on SYNGAP1 pathogenic mechanisms converge that de novo mutations are predominantly LoF alleles and lead to SYNGAP1 haploinsufficiency (Berryer et al., 2013). The development of treatment for SYNGAP1-associated conditions has a far-reaching impact.

Syngap1 was initially identified by sequencing proteins in the post-synaptic density (PSD) or interacting with PDZ domains (Chen et al., 1998; Kim et al., 1998). Syngap1 suppresses ERK phosphorylation and surface AMPA receptor levels (Kim et al., 2005; Rumbaugh et al., 2006); phosphorylation of Syngap1 by CaM Kinase II rapidly disperses Syngap1 from synaptic spines and triggers AMPA receptor insertion (Araki et al., 2015; Chen et al., 1998). Homozygous Syngap1 knockout (Syngap1−/−) mice died within 48 hours after birth; heterozygous Syngap1 knockout led to ~50% reduction of Syngap1 protein, reduced long-term potentiation (LTP), impaired membrane excitability, decreased ability in spatial learning, and reduced cognition primarily due to defects in forebrain excitatory neurons (Kim et al., 2003; Komiyama et al., 2002; Michaelson et al., 2018; Ozkan et al., 2014). Loss of Syngap1 in GABAergic cells was also reported to impair cognitive functions (Berryer et al., 2016). Mechanistically, Syngap1 heterozygous knockout mice exhibited increased excitatory synaptic transmission in early postnatal development due to enhanced AMPA receptor sensitivity (Clement et al., 2012). The re-expression of Syngap1 in adult mice slightly improved brain functions and behaviors (Creson et al., 2019), implying that upregulating SYNGAP1 protein expression in the brain can potentially alleviate symptoms in human patients with SYNGAP1 haploinsufficiency.

Alternative splicing coupled with nonsense-mediated mRNA decay (AS-NMD), or unproductive splicing, is frequently used by RNA splicing regulators as a negative autofeedback for homeostatic protein expression (Black, 2003; Lareau et al., 2007). AS-NMD is predicted to regulate hundreds of genes in mammalian brains (Yan et al., 2015), but its role in human brain development has only started to be explored by us and others recently (Carvill et al., 2018; Zhang et al., 2016). In past years, splice-switching oligos (SSOs) targeting genes such as SMN2 have been successful in treating neurological disorders (Finkel et al., 2017; Hua et al., 2011), paving the path for SSO-mediated therapy and personalized medicine (Kim et al., 2019). Thus, there is a need to develop specific, non-toxic ASOs (including SSOs) for mediating SYNGAP1 alternative splicing.

### BRIEF SUMMARY

In general, the current invention relates to the discovery that the design of antisense oligonucleotides (ASOs), which include SSOs, that bind a SYNGAP1 pre-mRNA is critical for blocking alternative splicing that leads to an efficient increase of protein produced from matured SYNGAP1 mRNA. In particular, the inventors have determined that ASOs that bind multiple regions of their target molecule are less effective than the new ASOs described herein. The desired increase in protein can alleviate issues related to loss-of-function mutations in the SYNGAP1 gene, including those that lead to haploinsufficiencies. These significant improvements can be useful to treat neurological disorders that result from such mutations.

Certain aspects relate to methods of increasing a SYNGAP1 protein in a cell. In some aspects, the SYNGAP1 protein is produced from a SYNGAP1 pre-mRNA. In certain aspects, the SYNGAP1 protein is increased in the cell by contacting the cell with an ASO. The ASO may be any ASO described herein. The cell may be any cell that expresses the SYNGAP1 pre-mRNA. In some aspects, the cell is a neuron. The cell may comprise a haploinsufficiency of the SYNGAP1 gene. The cell may comprise a mutation in the SYNGAP1 gene, which may be a loss-of-function mutation. Certain aspects relate to methods where the cell is contacted with an ASO. In some aspects, the cell is contacted with 1, 2, 3, 4, 5, 6, 7, 8, 9, 10, or more (or any range derivable therein) different ASOs each comprising a unique sequence.

In some aspects, the ASO is fully complementary to only one contiguous region of the SYNGAP1 pre-mRNA. The one contiguous region may be an intronic region of the SYNGAP1 gene, such as a region in intron 10 of the SYNGAP1 gene. The one contiguous region may be an alternative splice site in the SYNGAP1 gene. The one contiguous region may be in an alternatively spliced exon, i.e. an exon that comprises a different 5′ or 3′ sequence depending on which splice site is used during pre-mRNA processing. In some aspects, the alternatively spliced exon comprises an A3SS of the SYNGAP1 gene. The one contiguous region may be in intron 10 of the SYNGAP1 gene. The one contiguous region may be an alternative splice site (which may be in an alternatively spliced exon) in intron 10 of the SYNGAP1 gene. The one contiguous region may be a binding site for one or more proteins involved in splicing. The one contiguous region may be a binding site for a polypyrimidine tract binding protein (PTBP). The one contiguous region may comprise a motif for one or more proteins involved in splicing, such as PTBP. In some aspects, the one contiguous region comprises all or part of a PTBP-motif, which may be in intron 10 of the SYNGAP1 pre-mRNA. It is specifically contemplated that, in certain aspects, the ASO does not bind in more than one contiguous region to the pre-mRNA. It is also specifically contemplated that, in certain aspects, the ASO is not complementary to a sequence in the pre-mRNA that is not proximal to an alternative splice site.

In some aspects, the ASO has at least 90%, 95%, or 100% sequence identity to SEQ ID NO:1. In some aspects, the ASO has at least 90%, 95%, or 100% sequence identity to SEQ ID NO:2. In some aspects, the ASO has at least 90%, 95%, or 100% sequence identity to SEQ ID NO:3. In some aspects, the ASO has at least 90%, 95%, or 100% sequence identity to SEQ ID NO:4. In some aspects, the ASO has at least 90%, 95%, or 100% sequence identity to SEQ ID NO:5. In some aspects, the ASO has 15, 16, 17, 18, 19, 20, 21, 22, 23, 24, or 25 (or any range derivable therein) nucleotides. In certain aspects, the ASO is isolated. In some aspects, the ASO consists of an ASO with at least 90%, 95%, or 100% sequence identity to SEQ ID NO:1, SEQ ID NO:2, SEQ ID NO:3, SEQ ID NO:4, or SEQ ID NO:5.

In some aspects, the ASO has 1, 2, 3, 4, 5, 6, 7, 8, 9, 10 or more (or any range derivable therein) nucleotides inserted, contiguously or separately, into the sequence of SEQ ID NO:1, SEQ ID NO:2, SEQ ID NO:3, SEQ ID NO:4, or SEQ ID NO:5. The insertions may be on the 5′ and/or 3′ end of the sequence, and/or be inserted within the sequence. In some aspects, the ASO has 1, 2, 3, 4, 5, 6, 7, 8, 9, 10 or more (or any range derivable therein) mutations to SEQ ID NO:1, SEQ ID NO:2, SEQ ID NO:3, SEQ ID NO:4, or SEQ ID NO:5. In some aspects, the ASO has 1, 2, 3, 4, 5, 6, 7, 8, 9, 10, or more (or any range derivable therein) deletions to SEQ ID NO:1, SEQ ID NO:2, SEQ ID NO:3, SEQ ID NO:4, or SEQ ID NO:5.

In certain aspects, the ASO consists of an ASO with at least 90%, 95%, or 100% sequence identity to SEQ ID NO:1, SEQ ID NO:2, SEQ ID NO:3, SEQ ID NO:4, or SEQ ID NO:5. In certain aspects, the ASOs consist of or comprise two or more ASOs having at least 90%, 95%, or 100% sequence identity to SEQ ID NO:1, SEQ ID NO:2, SEQ ID NO:3, SEQ ID NO:4, or SEQ ID NO:5. In some aspects, one or more ASO, including one or more ASO described herein, is specifically excluded from the method.

Certain methods herein reduce and/or prevent nonsense-mediated decay of an mRNA derived from a SYNGAP1 pre-mRNA. In some aspects, the SYNGAP1 pre-mRNA can be processed into an mRNA, where the mRNA has a stop codon upstream of a splicing junction forming a nonsense mRNA. In such aspects, the nonsense mRNA undergoes nonsense-mediated decay. In certain aspects, the ASO, having complementary to one contiguous region of the SYNGAP1 pre-mRNA, blocks the processing of the SYNGAP1 pre-mRNA into the nonsense mRNA. In certain aspects, the ASO specifically binds to a SYNGAP1 pre-mRNA that gets processed into the nonsense mRNA, but does not bind to a SYNGAP1 pre-mRNA that does not get processed into the nonsense mRNA. In some aspects, the ASO binds to productive pre-mRNA (pre-mRNA that does not get processed into nonsense mRNA) and binds to unproductive pre-mRNA (pre-mRNA that does get processed into nonsense mRNA). In certain aspects, the ASO blocks at least one sequence that are required to generate unproductive mRNA. The ASO may block one or more proteins involved in splicing, including a polypyrimidine tract binding protein (PTBP). In some aspects, a cell is contacted with an amount of the ASO sufficient to reduce nonsense-mediated decay of an mRNA derived from the SYNGAP1 pre-mRNA, which may be the nonsense mRNA. The reduction may be at least, or approximately equal to, a 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any range derivable therein) reduction in nonsense-mediated decay of SYNGAP1 mRNA in the cell compared to a cell not contacted with the ASO. In some aspects, the cell is contacted with an amount of the ASO sufficient to increase the protein produced from the SYNGAP1 pre-mRNA to an amount that is more than, or approximately equal to, a 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0-fold, or any range derivable therein, greater than an amount of protein produced from the SYNGAP1 pre-mRNA present in a cell not contacted with the ASO.

The SYNGAP1 protein may be any isoform. In some aspects, the SYNGAP1 protein is a SYNGAP1 isoform expressed in neurons. In some aspects, the SYNGAP1 protein is produced from a SYNGAP1 mRNA that comprises, consisting essentially of, or consists of one or more of exons 1, 2, 3, 10, 11, 13, and 14 of the SYNGAP1 gene. The SYNGAP1 protein may be produced from a SYNGAP1 mRNA that comprises, consists essentially of, or consists of exons 1, 2, 3, 4, 5, 6, 7, 8, 9, 10, 11, 12, 13, 14, 15, 16, 17, 18, and/or 19, or any combination thereof, of the SYNGAP1 gene.

Certain aspects relate to methods comprising administering a therapeutically effective amount of at least one ASO to a patient. In certain aspects, the ASO consists of or comprises one or more ASOs disclosed herein. In certain aspects, the ASO consists of an ASO with at least 90%, 95%, or 100% sequence identity to SEQ ID NO:1, SEQ ID NO:2, SEQ ID NO:3, SEQ ID NO:4, or SEQ ID NO:5. In certain aspects, the ASOs consist or comprise two or more ASOs having at least 90%, 95%, or 100% sequence identity to SEQ ID NO:1, SEQ ID NO:2, SEQ ID NO:3, SEQ ID NO:4, or SEQ ID NO:5. In some aspects, one or more ASO, including one or more ASO described herein, is specifically excluded from the method.

In certain aspects, the method comprising administering the ASO is a method of treating the patient, such as treating a neurological disorder in the patient. The neurological disorder may be autism, an intellectual disability, and/or epilepsy. The intellectual disability may be an intellectual developmental disability, including for example autosomal dominant 5 (MRD5, OMIM code 612621, 2023 ICD-10-CM code F78. A1) The neurological disorder may be any one of attention deficit hyperactivity disorder, autism, Asperger syndrome, Tourette's syndrome, obsessive compulsive disorder, one or more neurobehavioral associated symptoms of degeneratives of the nervous system, Parkinson's disease, essential tremor, Huntington's disease, Alzheimer's disease, multiple sclerosis, or organic psychosis. In certain aspects, the method comprising administering the ASO is a method of reducing symptoms or pathologies associated with reduced SYNGAP1 protein levels, including those associated with reduced SYNGAP1 protein levels in a neuron in the patient. In some aspects, the reduced protein levels are due to a loss-of-function mutation and/or haploinsufficiency of the SYNGAP1 gene in the cell. In some aspects, the loss-of-function mutation in the SYNGAP1 gene causes nonsense-mediated decay of mRNA transcribed from the SYNGAP1 gene absent the ASO contacting the cell.

Certain aspects relate to the compositions described herein, such as any of the ASOs described herein. In certain aspects, the ASO, which may be isolated, comprises a sequence complementary to one contiguous region of a SYNGAP1 pre-mRNA, wherein the ASO is capable of binding to the contiguous region of the SYNGAP1 pre-mRNA in a manner that blocks an alternative splicing of the SYNGAP1 pre-mRNA that leads to nonsense-mediated decay of an mRNA derived from the SYNGAP1 pre-mRNA. In certain aspects, the ASO comprises SEQ ID NO: 1. In certain aspects, the ASO comprises SEQ ID NO:2. In certain aspects, the ASO comprises SEQ ID NO:3. In certain aspects, the ASO comprises SEQ ID NO:4. In certain aspects, the ASO comprises SEQ ID NO:5. In certain aspects, the composition consists of an ASO with at least 90%, 95%, or 100% sequence identity to SEQ ID NO:1, SEQ ID NO:2, SEQ ID NO:3, SEQ ID NO:4, or SEQ ID NO:5. In certain aspects, the composition consists of or comprises two or more ASOs having at least 90%, 95%, or 100% sequence identity to SEQ ID NO:1, SEQ ID NO:2, SEQ ID NO:3, SEQ ID NO:4, or SEQ ID NO:5. In some aspects, one or more ASO, including one or more ASO described herein, is specifically excluded from the composition. In certain aspects, the composition comprises one or more ASOs, including one or more ASOs disclosed herein, where none of the ASOs bind more than once to a pre-mRNA. It is also specifically contemplated that, in certain aspects, the SYNGAP1 pre-mRNA does not contain more than binding site to the specific ASO or ASOs in the composition.

Certain aspects relate to pharmaceutical compositions comprising at least one ASO. The pharmaceutical composition may comprise any ASO described herein. In certain aspects, the pharmaceutical composition consists of an ASO with at least 90%, 95%, or 100% sequence identity to SEQ ID NO:1, SEQ ID NO:2, SEQ ID NO:3, SEQ ID NO:4, or SEQ ID NO:5 and a pharmaceutical excipient. In certain aspects, the pharmaceutical composition consists of or comprises two or more ASOs having at least 90%, 95%, or 100% sequence identity to SEQ ID NO:1, SEQ ID NO:2, SEQ ID NO:3, SEQ ID NO:4, or SEQ ID NO:5. In some aspects, one or more ASO, including one or more ASO described herein, is specifically excluded from the pharmaceutical composition. In some aspects, the ASO in the pharmaceutical composition is isolated. The pharmaceutical composition may be formulated to stabilize the ASO. In certain aspects, a therapeutically effective amount of the ASO is administered to a patient, including a patient having a neurological disorder. In certain aspects, the pharmaceutical composition comprises one or more ASOs, including one or more ASOs disclosed herein, where none of the ASOs bind more than once to a pre-mRNA. It is also specifically contemplated that, in certain aspects, the SYNGAP1 pre-mRNA does not contain more than binding site to the specific ASO or ASOs in the pharmaceutical composition.

The term “one contiguous region,” as used herein, refers to a contiguous, unique region in a nucleic acid, including a pre-mRNA. In some aspects, the one contiguous region is only found once in a pre-mRNA, and includes sequences that are complementary to CH933, CH937, CH937-L, CH937-S, and/or YRW266. For example, SEQ ID NO:1 comprises a sequence complementary to one contiguous region that occurs only once in a SYNGAP1 pre-mRNA. In other aspects, there are contiguous regions in the pre-mRNA that are repeated, including those that are complementary to previously known ASOs. It is specifically contemplated that, in certain aspects, at least one ASO does not bind to more than one contiguous region.

Throughout this application, the term “about” is used according to its plain and ordinary meaning in the area of cell and molecular biology to indicate that a value includes the standard deviation of error for the device or method being employed to determine the value.

As used herein, “isolated” means altered or removed from the natural state through human intervention. For example, an ASO, such as an ASO comprising SEQ ID NO:1 or SEQ ID NO:2, naturally present in a living animal is not “isolated,” but a synthetic ASO, or an ASO partially or completely separated from the coexisting materials of its natural state is “isolated.” An isolated ASO can exist in substantially purified form, or can exist in a non-native environment such as, for example, a cell into which the ASO has been delivered.

As used herein, the terms “therapeutic composition,” “pharmaceutical composition,” “therapeutic agent” and “pharmaceutical agent” may be used interchangeably and refer to a composition that is used therapeutically to affect a response in a patient.

The use of the word “a” or “an” when used in conjunction with the term “comprising” may mean “one,” but it is also consistent with the meaning of “one or more,” “at least one,” and “one or more than one.” Any term used in singular form also comprises plural forms and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

It is contemplated that any aspect discussed in this specification can be implemented with respect to any method or composition of the invention, and vice versa. Furthermore, compositions of the invention can be used to achieve methods of the invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also aspects that may be implemented in the context of aspects discussed elsewhere in a different Example or elsewhere in the application, such as in the Summary of Invention, Detailed Description, Claims, and Brief Description of the Drawings.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific aspects presented herein.

FIGS. 1A-1G show alternative 3′ splice site of mouse Syngap1 inton10 induces nonsense-mediated mRNA decay. (A) Sashimi plots of isolated cortical neurons and apical neural progenitors showing Syngap1 A3SS in embryonic day 14.5 (E14.5) mouse dorsal forebrain. The A3SS exon inclusion ratios are indicated. (B) RNA-Seq results showing that Syngap1 A3SS was enriched in early brain development. (C) RT-PCR results showing that the Syngap1 A3SS was higher in the developing forebrain (76% at E12.5) and remained detectable in adulthood (5% at P40). PSI represents Percent Spliced In. One biological sample per lane. (D) Syngap1 A3SS introduces in-frame stop codons that truncate the protein and/or induce nonsense-mediated mRNA decay. (E) The predicted Syngap1 short protein isoform was not detectable in mouse brain lysates. (F) The Syngap1 A3 SS transcripts were enriched in Neuro2a cells treated with cycloheximide (CHX, two biological replicates per condition, p<0.001, unpaired t-test). (G) The Syngap1 A3SS-NMD was upregulated in Neuro2a treated with two siRNAs against Upf1 (adj.p<0.05 for siRNA-1, adj.p<0.01 for siRNA-2, one-way ANOVA). Two biological replicates per condition.

FIGS. 2A-2H show human SYNGAP1 A3SS induces nonsense-mediated mRNA decay in neural development. (A) RNA-Seq results showing SYNGAP1 A3SS in the laser microdissected cortical plate (CP) and ventricular zone (VZ) of gestational week 16 (GW16) fetal brains (Camp et al., 2015). (B) RT-PCR results showing that SYNGAP1 A3SS was enriched in fetal cortical development. (C) RT-PCR results showing that SYNGAP1 A3SS levels significantly decreased in iPSCs during NGN1/2-induced neuronal differentiation. Two biological replicates per condition, p<0.001, one-way ANOVA. (D) SYNGAP1 A3SS was enriched in iPSCs after CHX treatment. p<0.001 by t-test, three biological replicates. (E) SYNGAP1 A3SS ratio was increased in iPSC-derived neurons after CHX treatment. p<0.001 by t-test, three biological replicates. (F) Sequence alignment showing that the premature stop codon (TGA) in SYNGAP1 A3SS is conserved in mammals while the splice acceptor sites (AG) are variable. The AG sites were annotated according to RNA-Seq results of corresponding species in NIH Genome Data Viewer. (G-H) RT-PCR results of SYNGAP1 mini-gene constructs (G) transfected in Neuro2a cells showing that human SYNGAP1 intronic mutations identified in autism and ID patients led to intron 10 retention (c.1676+5 G>A, NM_006772.2) or abnormal A3SS inclusion (c.1677-2_1685del, p<0.001 by t-test, three biological replicates H).

FIGS. 3A-3G show SYNGAP1 A3SS-NMD is regulated by PTBP proteins. (A) Western blot results showing shRNA knockdown of Ptbp1 and Ptbp2 in Neuro2a cells. Two different shRNAs were used for each of Ptbp1 and Ptbp2. (B) RT-PCR results showing that loss of Ptbp1/2 promoted splicing of the canonical/productive Syngap1 isoform. (C) Immunofluorescence staining of primary cortical neurons (DIV5) showing that ectopic expression of PTBP1 (red, mCherry) decreased Syngap1 protein level (green). (D) CLIP-Seq analyses showing Ptbp1, Ptbp2 and U2af65 binding peaks in the A3SS region. (E) Mini-gene constructs of the human SYNGAP1 showing deletion of predicted splicing elements, and RT-PCR results showing their effects on A3SS insertion. Noticeably, the intronic element #1 and predicted upstream U2AF65-PTBP binding site #1 were required for A3SS inclusion; the predicted U2AF65-PTBP binding site #2 was required for canonical splicing and A3SS skipping. Two biological replicates for each condition. (F) EMSA assay showing that PTBP1 protein had a higher affinity to the PTBP binding site #2 RNA probe. (G) Current working model: in neural progenitors and differentiating neurons, PTBP proteins bind to site #2 in A3SS (red) and suppress the canonical/neuronal 3′ splice site; in neurons, PTBP proteins are turned down/off, so site #2 is exposed for splicing machinery (U2AF65) recognition and promotes neuronal isoform expression.

FIGS. 4A-4I show genetic deletion of Syngap1 A3SS-NMD increases Syngap1 protein in the neocortex. (A) CRISPR deletion of Syngap1 A3SS-NMD in mice to generate the Syngap1 NISO (N) allele (chr17:26959184-26959451, mm10) and the short NISO allele (S, chr17:26959185-26959353, mm10). (B-C) RT-PCR results showing that Syngap1 A3SS-NMD exon11 was included in wild-type controls (+/+), decreased in Syngap1-NISO heterozygotes (N/+), and excluded in Syngap1-NISO homozygotes (N/N) in P1 (B) and P10 (C) dorsal cortices. (D) Western-blot results showing Syngap1 protein levels in P2 cortices. (E) Quantification of Syngap1 Western bot signals in D) relative to Gapdh showing that Syngap1 levels were significantly increased in Syngap1 N/+(32±9%, p<0.005) and Syngap1 N/N (58+/−6%, p<0.001, one-way ANOVA, Dunnett's multiple comparison test) when compared with wild-type (+/+). (F) fEPSP recordings of adult Syngap1 N/+ and N/N mice. Numbers of animals and slices for each genotype were: +/+(n=6 animals, 11 slices), N/+(n=6, 13), N/N (n=5, 11). *, adj.p=0.0223. (G-I) Barnes maze test results showing similar performance between wild-type (+/+, n=6) and mutants (N/+, n=7; N/N, n=6). (G) All genotypes show improvement in the primary latency to the exit zone over the course of training. (H) Comparisons of primary latency to the exit zone during the probe trial shows that Syngap1 mutant mice performed similarly to wild-type mice. (I) Heat maps and a box plot showing entry probability to the exit zone (E) and each false exit during the probe trial. Syngap1 N/+ and Syngap1 N/N animals were not significantly different in behavior from wild-type (+/+) mice (adj.p>0.05). The greatest entry probability for each genotype corresponds to the exit zone.

FIGS. 5A-5D show genetic deletion of Syngap1 A3SS-NMD alleviates LTP and membrane excitability deficits caused by a conditional Syngap1 knockout allele. (A) fEPSP recordings showing that the N allele alleviates the LTP deficit in P35-40 Syngap1 cKO mice. Numbers of animals and slices for each genotypes are: WT (replotted from FIG. 4F), cKO/+(n=5, 12), and cKO/N (n=6, 12). ****adj.p<0.0001. (B) Typical traces from L2/3 pyramidal neurons in S1 cortex in WT (top row), cKO/+(middle row) and cKO/N mice (bottom row). Left to right: traces at current steps ~200 pA (left), ~400 pA (middle), and ~600 pA (right). (C) Spike count (number of evoked spikes) per current step for WT (n=8), cKO/+(n=8) and cKO/N mice (n=7). The average±SD is shown for all data points. p=0.0003, F (2, 177)=8.420 for genotype, two-way ANOVA. (D) Dot plots showing the maximal spike frequency obtained from the same recordings. p=0.0007 by one-way ANOVA. Each dot represents one animal. The average SD is shown for all data points.

FIGS. 6A-6Q show the lead SSO upregulates SYNGAP1 expression in human iPSCs and iPSC-derived neurons. (A) Schematic illustration of the SSO design targeting the SYNGAP1 A3SS. (B) RT-PCR results showing the screening of SSOs in iPSCs (PGP1-iNGN). One biological sample per lane. (C-E) Identification of the lead SSO in iPSC-derived neurons. RT-PCR results (C) and quantification (D) showing that CH937 suppresses SYNGAP1 A3SS in iPSC-derived neurons. Q-PCR results (E) showing that the productive SYNGAP1 transcript was upregulated in CH937-treated human iPSC-derived neurons. (F-K) The lead SSO suppresses SYNGAP1 A3SS in two additional human iPSC lines. RT-PCR results (F, I) and quantification (G, J) showing that CH937 suppressed SYNGAP1 A3SS in human iPSCs (NA19101 and 28126). Q-PCR results (H, K) showing that the CH937 significantly increased the productive SYNGAP1 transcript levels in human iPSC lines. (L-N) The lead SSO suppresses SYNGAP1 A3SS-NMD in SYNGAP1 patient-derived iPSCs. RT-PCR results (L) and quantification (M) showing that CH937 suppressed SYNGAP1 A3SS in SYNGAP1 patient-derived iPSCs (333de1, Lys114SerfsX20). Q-PCR results (N) showing that the CH937 significantly increased the productive SYNGAP1 mRNA level. (O-Q) Application of CH937 to human iPSC-derived cerebral organoids (O) led to increased SYNGAP1 protein expression (83% 28%, P-Q). p<0.05 by unpaired t-test.

FIGS. 7A-7C show Syngap1 A3SS induces nonsense-mediated mRNA decay. (A) RT-PCR results showing that the Syngap1 A3 SS inclusion in cultured primary cortical neurons. DIV, day cultured in vitro. PSI represents Percent Spliced In. (B) RT-PCR results showing that the Syngap1 A3SS inclusion was increased in primary cortical neurons after CHX treatment at DIV5 or DIV11. (C) siRNA targeting Upf1 significantly decreased Upf1 mRNA to 16.8% of the control in Neuro2a cells (p<0.001).

FIGS. 8A-8B show SYNGAP1 A3SS induces nonsense-mediated mRNA decay. (A) Human SYNGAP1 A3SS introduces premature stop codons (*). (B) SYNGAP1 A3SS-NMD decreased and remained detectable during NGN1/2-induced neuronal differentiation of iPSCs. p<0.0001 by one-way ANOVA.

FIGS. 9A-9D show PTBP proteins suppress SYNGAP1 expression by promoting A3SS-NMD. (A) Illustration of positions and coordinates (hg38) of SYNGAP1 A3SS, EMSA probes, PTBP binding motifs, U2AF2 CLIP-Seq tags, minigene deletions, and predicted sequences/sites for splicing regulation. (B) SYNGAP1 A3SS-NMD exon inclusion ratio (PSI, black line) and PTBP1/2 expression levels decreased during human brain development while the SYNGAP1 mRNA level increased. Re-analyzed RNA-Seq data from HDBR (Lindsay et al., 2016). (C-D) Expression of PTBP1 (IRES-mCherry, red) in primary neurons (DIV3-DIV5) led to decreased Syngap1 protein levels (green) in the soma and dendrites. Unpaired t-test.

FIGS. 10A-10J show Genetic deletion of Syngap1 A3SS-NMD in mice. (A) Genotyping results of the Syngap1 N and S mutant alleles. (B-C) RT-PCR and quantification results showing that the S allele (deletion of an intronic sequence) significantly decreased the Syngap1 A3SS-NMD in mice. One-way ANOVA. (D-E) Western-blot results showing that Syngap1 protein levels were significantly increased in P2 hippocampi by the N allele. (F) Immunostaining of the E14.5 mouse dorsal forebrain showing comparable neural-progenitor (Sox2) and neuron (NeuN) populations between wild-type and Syngap1 NN mutants. (G) Western-blot results showing that Syngap1 protein was undetectable in hearts or lungs of E18.5 wild-type or Syngap1 N/+ mutants. (H) Rotarod test results showing that there was no significant difference between wild-type (+/+, n=11) and Syngap1-NISO mutant mice (N/+, n=11; N/N, n=6). (I) Representative traces (top) and the corresponding input-output curves of the fEPSP in area CA1 (bottom). There was no significant difference between genotypes: wild-type (+/+, n=3 animals, 5 slices), N/+(n=4, 7), N/N (n=4, 9), p>0.05 by two-way ANOVA. (J) Paired-pulse ratios of the fEPSP in area CA1 (at inter-pulse intervals of: 20, 50, 100, 300 and 500 milliseconds). Genotype did not have a significant effect on the paired-pulse ratio: wild-type (+/+, n=4 animals, 6 slices), N/+(n=3, 6), N/N (n=4, 9), p>0.05 by two-way ANOVA.

FIGS. 11A-11I show characterization of the compound heterozygous Syngap1 fl/N; Emx1-Cre mice. (A) Genotyping results for the Syngap1 floxed cKO allele (fl). (B) Western blot results showing that Syngap1 was decreased in Syngap1 fl/+; Emx1-Cre animals (cKO/+). (C) A breeding scheme to generate excitatory neuron-specific Syngap1 deletion in the dorsal forebrain with the Emx1-Cre driver line, and the generation of compound heterozygous Syngap1 fl/N; Emx1-Cre animals. (D) Western blot results showing that Syngap1 was increased in cKO/N than in cKO/+ animals. (E) Representative traces of the fEPSP (top) and the corresponding input-output curves from area CA1 showing similar input-output relationships among the individual genotypes (bottom, p>0.05 by two-way ANOVA). Wild-type (+/+, n=3 animals, 5 slices; data replotted from FIG. 10I), cKO/+(n=4, 8), cKO/N (n=4, 11). (F) Paired-pulse ratios of the fEPSP in area CA1 (at interpulse intervals of: 20, 50, 100, 300 and 500 milliseconds). Genotype did not have a significant effect on the paired-pulse ratio (p>0.05 by two-way ANOVA). Numbers of animals and slices for each genotype are: wild-type (+/+) (n=4 animals, 6 slices; data replotted from FIG. 10J), cKO/+(n=4, 8), cKO/N (n=4, 7). (G) There was no significant difference in the action potential amplitude between WT (n=8 animals), Syngap1. cKO/+(n=8) or cKO/N (n=7) mice. All data are shown as average±SD. p=0.1934, one-way ANOVA. (H) There was no significant difference in the spike threshold between WT (n=8), Syngap1 cKO/+(n=8), or cKO/N (n=7) mice. All data are shown as average±SD. p=0.5834, one-way ANOVA. (I) There was no significant difference in the spike width at half amplitude between WT (n=8 animals), Syngap1 cKO/+(n=8), or cKO/N (n=7) mice. All data are shown as average±SD. p=0.2944, one-way ANOVA.

FIGS. 12A-12G show the lead SSO upregulates SYNGAP1 expression in human iPSCs and cerebral organoids. (A) Illustration of positions and coordinates (hg38) of SYNGAP1 SSOs, EMSA probes, PTBP binding motifs, U2AF2 CLIP-Seq tags, and predicted cis-regulatory elements. (B-C) RT-PCR results (B) and quantification (C) showing that CH937 suppressed SYNGAP1 A3SS in human iPSCs (PGP1-iNGN). Adjusted pairwise p value (NT vs CH937, one-way ANOVA) was shown. (D) Q-PCR results showing that the CH937 significantly increased the productive SYNGAP1 transcript in human iPSCs. The adjusted pairwise p value (NT vs CH937, one-way ANOVA) was shown. (E-G) Application of CH937 to human iPSC-derived cerebral organoids (E) led to increased SYNGAP1 protein expression (51%±10%, F-G). Adjusted pairwise p values are indicated (one-way ANOVA).

FIGS. 13A-13B show CH937 has higher efficiency than CH933 and ASO71 in repressing the SYNGAP1 A3SS-NMD and increasing productive transcripts in neurons. (A) Representative images of hiPSC iNGN and iNGN-derived neurons. The neurons were induced by treating iNGN with doxycycline hyclate for 4 days. (B) Agarose gel running results showing the dynamic changes of SYNGAP1 unproductive and productive transcripts during neuronal induction. NMD-in PSI means the percentage of NMD-transcript. Non-NMD transcript (productive) increased as induction went on.

FIG. 14 show CH937 and CH933 have lower toxicity to neurons than ASO71. A. Representative images of neurons 48 hours after ASOs treatment.

FIG. 15 shows the binding sites of ASO's (CH933, CH937, Lim71, and ET-019) within the SYNGAP1 mRNA seq. SnapGene software was used to produce the image showing three additional potential binding sites of Lim71 and two additional potential binding sites for ET-019. There are no additional binding sites for CH933 or CH937 identified.

FIGS. 16A-16D show ASOs screening and testing in SH-SY5Y cells. (A) RT-PCR results showed that SYNGAP1 intron 10 A3SS was enriched in SH-SY5Y cells after cycloheximide (CHX) treatment. P<0.0001 by t-test, with three biological replicates. (B) RT-PCR results showed the efficacy of three kinds of transfection reagents: jetOPTIMUP, Mirus, and Lipofectamine 3000. jetOPTIMUS performed better than Mirus and Lipofectamine 3000. (C) ASOs screening in SH-SY5Y by jetOPTIMUS transfection method showed that CH937 and CH937-S had better efficacy than others in reducing NMD-exon inclusion, as indicated by PSI values. SC937 represents the scramble ASO (18-nucleotide) targeting nothing as a control group. CH937 has 18 nucleotides. CH937-L represents the more extended version of CH937, with 21 nucleotides. CH937-S represents the shorter version of CH937, with 17 nucleotides. ET019 and ET085 are two ASOs used in a published study (PMID: 37149717). (D) Low-concentration testing of ASOs in SH-SY5Y cells by jetOPTIMUS transfection method. All the tested ASOs were effective in reducing the NMD-exon inclusion. At 50 nM in SH-SY5Y cells, no significant difference in efficacy was found between these ASOs. **, P<0.01; ***, P<0.001; ****, P<0.0001. One-way ANOVA test.

FIG. 17 shows the annotations of certain ASOs disclosed herein both upstream and within the SYNGAP1 NMD-exon. Lim71 was used in a published study (PMID: 32647108). ET019 and ET085 are two ASOs used in another published study (PMID: 37149717).

### DETAILED DESCRIPTION

Aspects herein relate to characterizations of Syngap1 A3SS-NMD inclusion in brain development and regulatory mechanisms. Certain aspects relate to intronic sequences required for Syngap1 A3SS-NMD inclusion, which may be genetically deleted or blocked with at least one antisense oligonucleotide (ASO). The ASO may be a splice-switching oligonucleotide (SSO). Such deletions or blocking may result in skipping of A3SS-NMD and enrichment of the neuronal isoform. In some aspects, decreased A3SS-NMD inclusion leads to increased Syngap1 protein. Certain aspects relate to the functions of Syngap1 A3SS-NMD, including in vivo, and how the A3SS-NMD exon is a suitable target to rescue haploinsufficiency.

The Ras GTPase activating protein SYNGAP1 plays a central role in synaptic plasticity, and de novo SYNGAP1 mutations are among the most frequent causes of autism, epilepsy, and intellectual disability. How SYNGAP1 is regulated during development and how to treat SYNGAP1-associated haploinsufficiency remain challenging questions. Aspects herein characterize an alternative 3′ splice site (A3SS) of SYNGAP1 that induces nonsense-mediated mRNA decay (A3SS-NMD), including in mouse and human neural development. In some aspects, two intronic SYNGAP1 mutations cause loss-of-function through intron retention or abnormal A3SS-NMD inpatients affected by autism and intellectual disability. Certain aspects pinpoint the regulatory sequences and demonstrate that PTBP proteins directly bind to SYNGAP1 and promote A3SS inclusion. Aspects herein show genetic deletion of Syngap1 A3SS in mice can upregulate Syngap1 protein and alleviates the long-term potentiation and membrane excitability deficits caused by a Syngap1 knockout allele. In some aspects, a splice-switching oligonucleotide (SSO) efficiently converts SYNGAP1 unproductive isoform to a functional form, including in human iPSCs and iPSC-derived neurons. Aspects herein describe the dynamic regulation and in vivo function of SYNGAP1 unproductive splicing, the genetic rescue of a Syngap1 knockout allele in mice, and the development of an SSO to alleviate SYNGAP1-associated haploinsufficiency.

Alternative splicing coupled with NMD (AS-NMD) can selectively remove unproductive transcripts and has been reported to regulate the expression of neuronal genes (Carvill et al., 2018; Yan et al., 2015; Zhang et al., 2016; Zheng et al., 2012). The therapeutic potential of targeting AS-NMD with SSOs has evoked unprecedented enthusiasm, and SSO screens in cultured cells have been reported in recent studies (Han et al., 2020; Lim et al., 2020). Certain aspects show that it may be safe to target naturally occurring AS-NMD for therapy. Certain aspects relate to whether an AS-NMD exon is required for normal physiological functions in animal development and whether it is safe to suppress or completely block such an AS-NMD exon in vivo. It is important to understand the in vivo expression patterns, regulatory mechanisms, and organismal functions of AS-NMD exons.

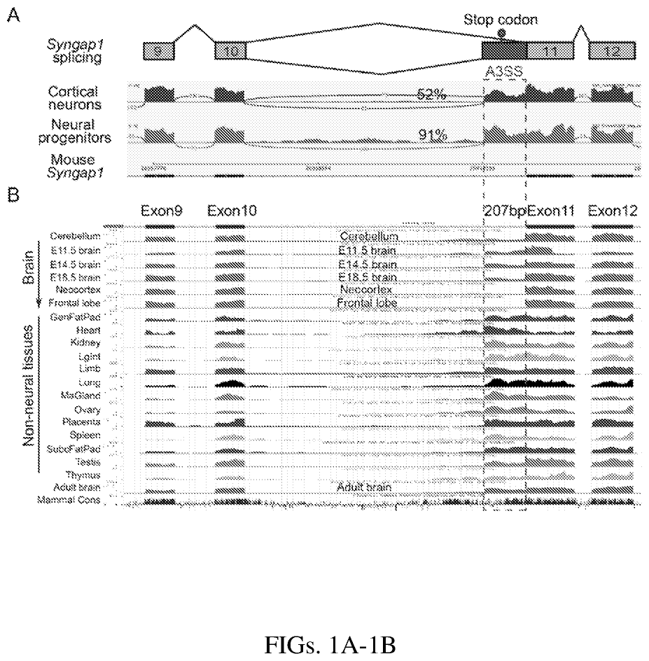

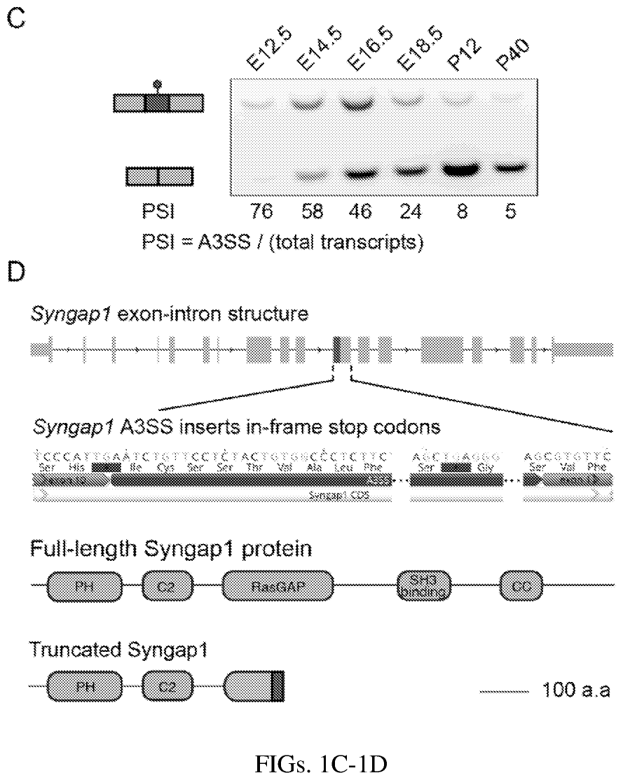

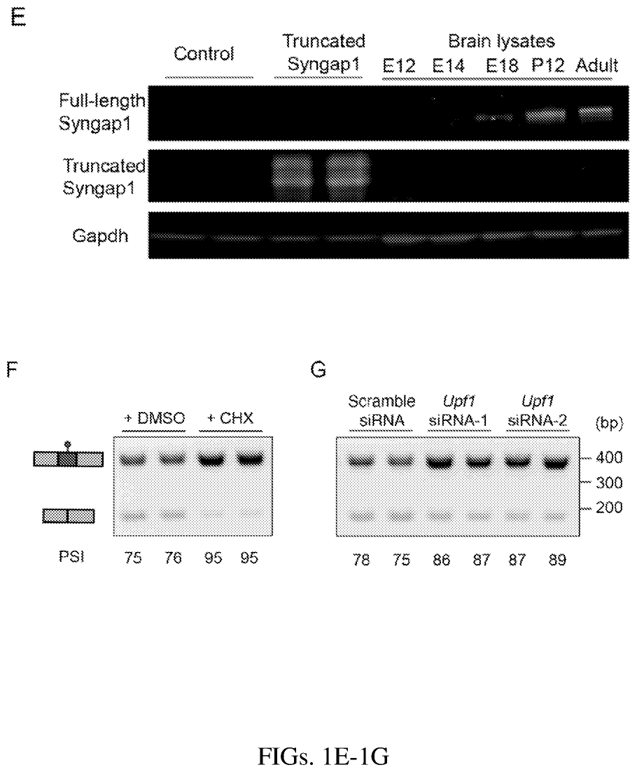

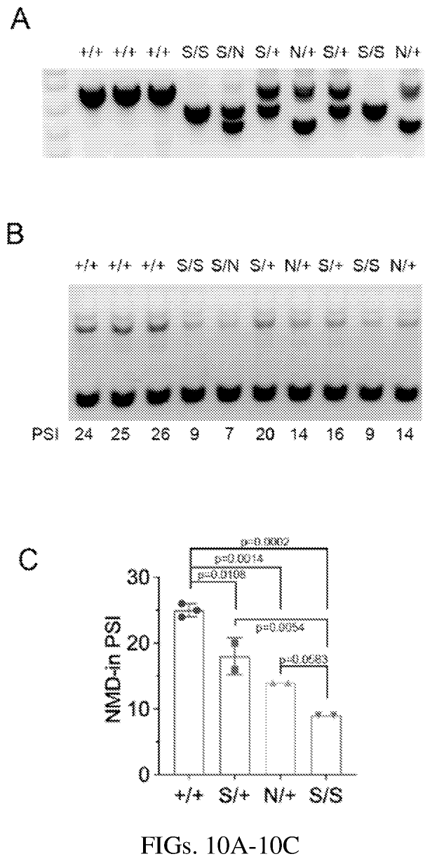

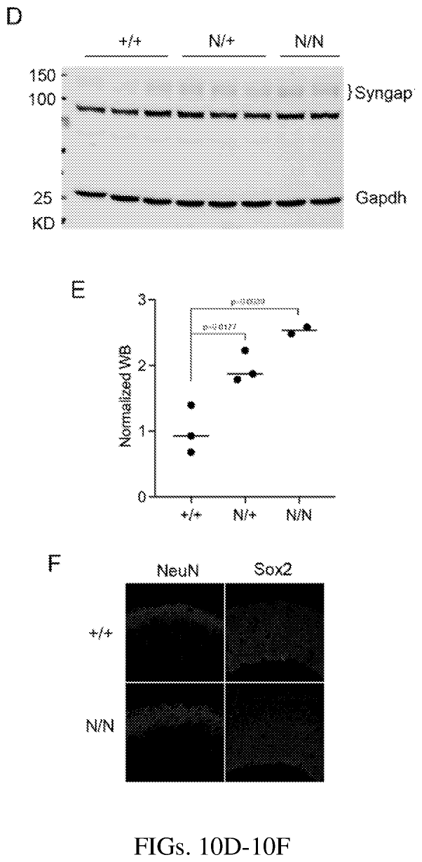

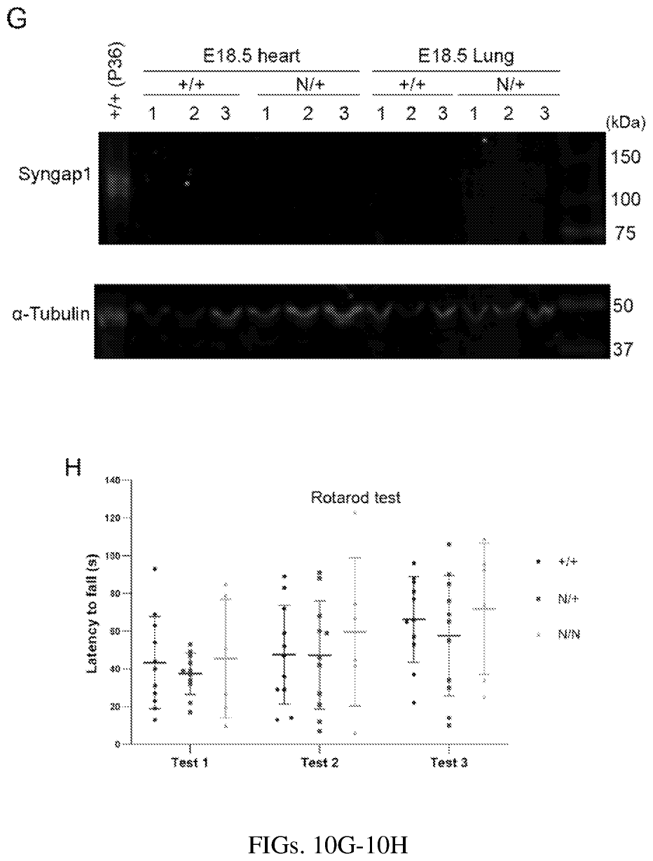

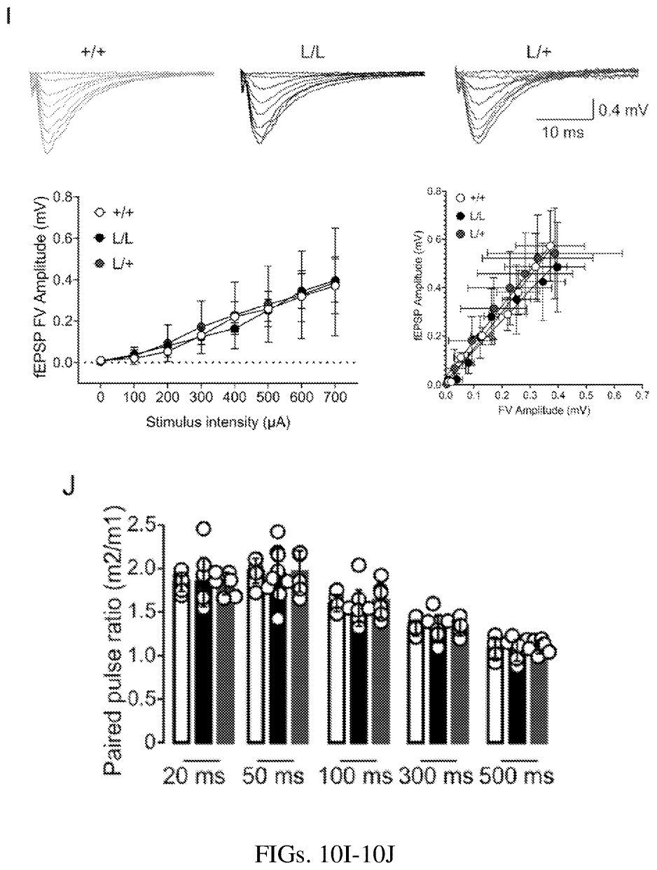

Certain aspects explore the function and therapeutic potential of the SYNGAP1 A3SS, including in mouse models and human iPSCs. Syngap1 is a synaptic protein barely detected in non-neuronal tissues (Kim et al., 1998). In contrast, the SYNGAP1 transcript is detectable in non-neural tissues in mice and humans (FIG. 1 and Genotype-Tissue Expression (GTEx)), where the Syngap1 A3SS-NMD inclusion is nearly constitutive (FIG. 1). These observations suggest that A3SS-NMD provides an orthogonal mechanism in non-neuronal cells to suppress excess or leaky SYNGAP1 expression which would waste cellular resources and interfere with Ras signaling. Aspects herein support that heterozygous genetic deletion of Syngap1 A3SS in mice upregulated SYNGAP1 protein during brain development. While homozygous deletion of mouse Syngap1 A3SS led to a modest reduction in the magnitude of long-term potentiation (LTP), it did not overtly impact behavioral performance in the Barnes maze or Rotarod assays. In certain aspects, Syngap1 protein is not detected in E18.5 heart and lung tissues from Syngap1 N/+ animals (FIG. 10G). In certain aspects, significant cortical neurogenesis defect in Syngap1 N/N mice (FIG. 10F) was not found. Certain aspects suggest that the Syngap1 A3SS is moderately required during neural development, and its functions in non-neural tissues remain to be further explored.

In certain aspects, heterozygous deletion of mouse Syngap1 A3SS alleviates the LTP deficits caused by a heterozygous Syngap1 knockout allele. In some aspects, suppressing A3SS-NMD alleviates Syngap1 haploinsufficiency, including in vivo. A previous study has reported that Syngap1 heterozygous deletion reduced membrane excitability, as tested via trains of injected current pulses of rising amplitude (Michaelson et al., 2018). Here, we adapted this experimental protocol to capture a cell physiological parameter that differs from LTP because it is cell autonomous. In some aspects, the maximal spike firing frequency (which, as a result of spike frequency adaptation occurs at the beginning of the current pulse (Gill and Hansel, 2020) and is typically determined from the interval of the first two spikes) is lower in Syngap1 heterozygous knockouts than in wild-type subjects. In some aspects, both the general excitability and the maximal spike frequency defects are alleviated by the heterozygous deletion of Syngap1 A3SS.

In some aspects, ASOs, including any SSO described herein such as CH937, effectively suppress SYNGAP1 A3SS, including in human iPSCs and iPSC-derived neurons, and significantly increased the functional SYNGAP1 isoform. In some aspects, the ASO, including SSO CH937, significantly increases SYNGAP1 protein expression, including in cerebral organoids induced from two different human iPSC lines.

In some aspects, a SYNGAP1 protein directly interacts with PSD-95 in the postsynaptic density, and remarkably, both genes undergo unproductive splicing that is promoted by PTBP1 and PTBP2 in early neural development. While PTBP proteins promote SYNGAP1 A3SS-NMD inclusion, they suppress the inclusion of a coding exon in PSD-95/DLG4 and lead to NMD (Zheng et al., 2012). Previous studies showed that protein levels of SYNGAP1 and PSD-95 exhibit a near stoichiometric ratio in the PSD and the appropriate protein ratio is critical for the formation of SYNGAP1-PSD-95 liquid-like droplets (Zeng et al., 2016). Strikingly, two AS-NMD events are co-regulated by the PTBP1/2 proteins for equilibrated protein expression. De novo mutations in PSD-95 have been reported to cause synaptopathy (Rodriguez-Palmero et al., 2021). In some aspects, the functions of the PSD-95 AS-NMD exon may function as a therapeutic target.

Hundreds of genes have been reported to undergo AS-NMD during cortical development, and chromatin regulators were highly enriched (Yan et al., 2015). Many of the AS-NMD exons are regulated by PTBP and RBFOX proteins, and mutations in the host genes are frequently associated with neurodevelopmental disorders such as autism and epilepsy (Carvill et al., 2018; Li et al., 2015; Vuong et al., 2016; Weyn-Vanhentenryck et al., 2014; Zhang et al., 2016). Although SSOs have been reported as promising ways to target AS-NMD and treat diseases such as the Dravet Syndrome (Han et al., 2020), the biological functions of such AS-NMD events were undetermined.

Certain aspects characterize the alternative splicing of SYNGAP1 intron 10, which can affect the size of exon11. In some aspects, the alternative splicing leads to NMD through an alternative 3′ splice site (A3SS-NMD), including in mouse and human brains. Certain aspects investigate the regulatory mechanisms and identify critical intronic elements essential for SYNGAP1 A3SS-NMD. Aspects of the disclosure concern one or more ASOs, which may be SSOs, such as CH933 and CH937, that are capable of suppressing SYNGAP1 A3SS-NMD. In some aspects, the ASOs can increase productive transcripts of SYNGAP1 in neurons. Certain aspects concern the use of the ASOs as treatments, including for neurological disorders (such as ASD, ID, epilepsy, etc.) caused by SYNGAP1 haploinsufficiency.

In some aspects, the ASOs disclosed herein, such as CH933 and/or CH937, are different from previously known ASOs. The ASOs disclosed herein, such as CH933 and CH937 have a higher efficiency (including a higher efficiency of increasing productive SYNGAP1 gene products) and/or lower toxicity compared to previously known ASOs. In some aspects, the ASOs disclosed herein, such as CH933 and CH937, lead to an increase of productive transcripts of SYNGAP1 encoding SynGAP protein, including when compared to previously known ASOs.

Aspects herein identify an alternative 3′ splice site (A3SS) of SYNGAP1 intron 10 that, in some circumstances, leads to NMD, including in mouse and human brain development. Alternative splicing, in some aspects, is a way to modulate SYNGAP1 protein expression, including in HEK293 cells (Lim et al., 2020). In some aspects, as a safety measure, it is essential to understand the organismal function and dosage effect of Syngap1 A3SS-NMD.

### I. Antisense Oligonucleotides

In some aspects, the disclosure relates to antisense oligonucleotides (ASOs) that inhibit the binding of certain splicing machinery, such as PTBP1 or PTBP2, which can affect the amount of a protein in a cell. In some embodiments, the ASO is a splice-switching oligonucleotide (SSO). In some aspects, the protein comprises SYNGAP1. In some aspects, the disclosure relates to expression systems capable of expressing the ASO. An ASO may increase the translation of a gene transcript in a cell. An ASO may be from 16 to 1000 nucleotides long, and in certain aspects from 15 to 100 nucleotides long. The ASO may have at least or may have at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, or 90 (or any range derivable therein) nucleotides. The ASO may comprise any nucleic acid form such as DNA, RNA, LNA, or BNA. The ASO may comprise synthetic or non-natural nucleotides. The ASO may be synthetic and/or isolated. In some aspects, the ASO is single-stranded. In some aspects, the ASO is double-stranded. In some aspects, the ASO comprises single-stranded DNA. In some aspects, the ASO comprises single-stranded RNA. In some aspects, the ASO comprises double-stranded DNA. In some aspects, the ASO comprises double-stranded RNA. In some aspects, the ASO comprises an antisense strand.

Particularly, an ASO may be capable of decreasing the nonsense-mediated decay of an mRNA by at least 10%, 20%, 30%, or 40%, more particularly by at least 50%, 60%, or 70%, and most particularly by at least 75%, 80%, 90%, 95%, 99%, or 100% more or any range or value in between the foregoing. In some aspects, the ASO is capable of increasing protein levels of a protein of interest, such as SYNGAP1, by modulating the splicing of a pre-mRNA transcript encoding the protein. The ASO may be partially or fully complementary to a sequence in the pre-mRNA that is involved in splicing, such as an alternative splicing site or a splicing factor-binding site.

In some aspects, the ASO is, or comprises, an oligonucleotide analog and may include modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

The present disclosure concerns modified oligonucleotides, i.e., oligonucleotide analogs or oligonucleosides, and methods for effecting the modifications. These modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages may replace naturally-occurring phosphodiester-5′-methylene linkages with four atom linking groups to confer nuclease resistance and enhanced cellular uptake to the resulting compound.

Modifications may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

Oligonucleosides linked by hydrazines, hydroxylarnines, and other linking groups, are contemplated herein for use in the ASOs, and can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc.® 380B and 394 and Milligen/Biosearch® 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass, TentaGel® Support—an aminopolyethyleneglycol derivatized support or Poros—a copolymer of polystyrene/divinylbenzene. Attachment and cleavage of nucleotides and oligonucleotides can be effected via standard procedures. As used herein, the term solid support further includes any linkers (e.g., long chain alkyl amines and succinyl residues) used to bind a growing oligonucleotide to a stationary phase such as CPG. In some aspects, the oligonucleotide may be further defined as having one or more locked nucleotides, ethylene bridged nucleotides, peptide nucleic acids, or a 5′(E)-vinyl-phosphonate (VP) modification. In some aspects, the oligonucleotides has one or more phosphorothioated DNA or RNA bases.

### II. Obtaining Nucleotides

### A. Synthesis

The nucleic acid molecules, including an ASO described herein, may be generated by nucleic acid synthesis. The ASOs may be synthesized using any method known in the art, such as phosphoramidite synthesis and/or solid-phase synthesis. The ASO analogs may be synthesized.

### B. Expression

The nucleic acid molecules, including any ASO described herein, may be generated by expression vectors. The expression vectors used herein may contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, and a selectable marker element. Such sequences and methods of using the same are well known in the art.

### 1. Expression Systems

Numerous expression systems exist that comprise at least a part or all of the expression vectors discussed above. Prokaryote- and/or eukaryote-based systems can be employed for use with an aspect to produce nucleic acid sequences. Commercially and widely available systems include but are not limited to bacterial, mammalian, yeast, and insect cell systems. Those skilled in the art are able to express a vector to produce a nucleic acid sequence using an appropriate expression system.

### 2. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 3. Host Cells

In another aspect, contemplated are the use of host cells into which a recombinant expression vector has been introduced. Vector DNA can be introduced into prokaryotic or eukaryotic cells via conventional transformation or transfection techniques. Some vectors may employ control sequences that allow it to be replicated and/or expressed in both prokaryotic and eukaryotic cells. One of skill in the art would understand the conditions under which to incubate host cells to maintain them and to permit replication of a vector. Also understood and known are techniques and conditions that would allow large-scale production of vectors, as well as production of the nucleic acids encoded by vectors.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### III. Administration of Therapeutic Compositions

The therapy provided herein may comprise the administration of one or a combination of therapeutic agents, such as one or a combination of unique antisense oligonucleotides (ASOs) and/or a combination of ASOs and other therapeutic compositions, including those useful for treating disorders disclosed herein, such as any neurological disorder, to a patient. In some aspects, the therapy is a cocktail of ASOs. In some aspects, the other therapeutic compositions are useful for reducing symptoms of the neurological disorder and/or reducing side effects of the other therapeutic agents administered. The therapies may be administered in any suitable manner known in the art. In some aspects, a first therapeutic composition (such as an ASO) and a second composition (such as another ASO or another therapeutic composition) may be administered sequentially (at different times) or concurrently (at the same time). In some aspects, the first and second therapeutic compositions are administered in a separate composition. In some aspects, the first and second therapeutic compositions are in the same composition.

In some aspects, the first therapeutic composition and the second therapeutic composition are administered substantially simultaneously. In some aspects, the first therapeutic composition and the second therapeutic composition are administered sequentially. In some aspects, the first therapeutic composition, the second therapeutic composition, and a third therapeutic composition are administered sequentially. In some aspects, the first therapeutic composition is administered before administering the second therapeutic composition. In some aspects, the first therapeutic composition is administered after administering the second therapeutic composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the therapy is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, a single dose of the ASO or other therapeutic composition is administered. In some aspects, multiple doses of the ASO or other therapeutic composition are administered. In some aspects, the ASO, at least one ASO, multiple ASOs, therapeutic compositions comprising one or more ASO, or other therapeutic composition is administered at a dose of between 1 mg/kg and 5000 mg/kg. In some aspects, the ASO, at least one ASO, multiple ASOs, therapeutic compositions comprising one or more ASO, or other therapeutic composition is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128,129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg, or any range derivable therein.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 day, week, month, or year intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### A. Pharmaceutical Compositions

In certain aspects, the compositions or agents, including those for use in the methods disclosed herein, such as one or more antisense oligonucleotides (ASOs), are suitably contained in a pharmaceutically acceptable carrier. The carrier can be non-toxic, biocompatible, and selected so as not to detrimentally affect the biological activity of the agent. The agents in some aspects of the disclosure may be formulated into preparations for local delivery (i.e. to a specific location of the body, such as the brain, nervous tissue, or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

Certain aspects disclosed herein concern compositions comprising a nanoparticle, which may encapsulate a therapeutic agent, which can be any of the therapeutic agents disclosed herein. The nanoparticle compositions may encapsulate therapeutic agents, which may be engineered protein compositions. The engineered proteins formulated in the nanoparticles can have improved pharmacokinetic and/or pharmacodynamic properties. The engineered proteins formulated in the nanoparticles are better tolerated by a patient, including a cancer patient. The engineered proteins formulated in the nanoparticles, in some aspects, are more effectively delivered to cells to effect their function, such as effecting transcriptional changes, than naked engineered proteins. In several aspects, the composition confers water solubility to hydrophobic agents, to combinations of hydrophobic agents, and/or to combinations of hydrophobic and hydrophilic agents. In several aspects, the nanoparticle composition comprises a liposomal and/or nano-emulsion composition of a therapeutic agent.

In several aspects, as disclosed elsewhere herein, a nanoparticle composition (e.g., a mixed micelle composition, a liposomal composition, solid lipid particles, oil-in-water emulsions, water-in-oil-in-water emulsions, water-in-oil emulsions, oil-in-water-in-oil emulsions, etc.) is provided to aid in the delivery of therapeutic agents. As disclosed elsewhere herein, in several aspects, the nanoparticles comprise one or more therapeutic agents. In several aspects, a composition comprising the nanoparticles disclosed herein comprises a therapeutically effective amount of one or more therapeutic agents. In several aspects, the nanoparticle composition (e.g., when in water or dried) comprises multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, and/or combinations thereof. In certain aspects, the nanoparticle compositions do not comprise multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, and/or combinations thereof. In several aspects, the composition is characterized by having multiple types of particles (e.g., lamellar, emulsion, irregular, etc.). In other aspects, a majority of the particles present are emulsion particles. In several aspects, a majority of the particles present are lamellar (multilamellar and/or unilamellar). In other aspects, a majority of the particles present are irregular particles. In still other aspects, a minority of the particles present are emulsion particles. In several aspects, a minority of the particles present are lamellar (multilamellar and/or unilamellar). In other aspects, a minority of the particles present are irregular particles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### B. Proteins

The nucleotides as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

### C. Other Agents

It is contemplated that other agents may be used in combination with certain aspects of the present aspects to improve the therapeutic efficacy of treatment. These additional agents include agents that act in combination and/or synergistically with the ASOs described herein. The additional agents may comprise agents that reduce symptoms of the disorders disclosed herein, or may comprise agents that reduce side effects associated with the therapeutic compositions disclosed herein.

### EXAMPLES

The following examples are included to demonstrate certain aspects of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the disclosure.

### Example 1: Alternative Splicing of Mouse Syngap1 Intron10 Leads to Nonsense-Mediated mRNA Decay

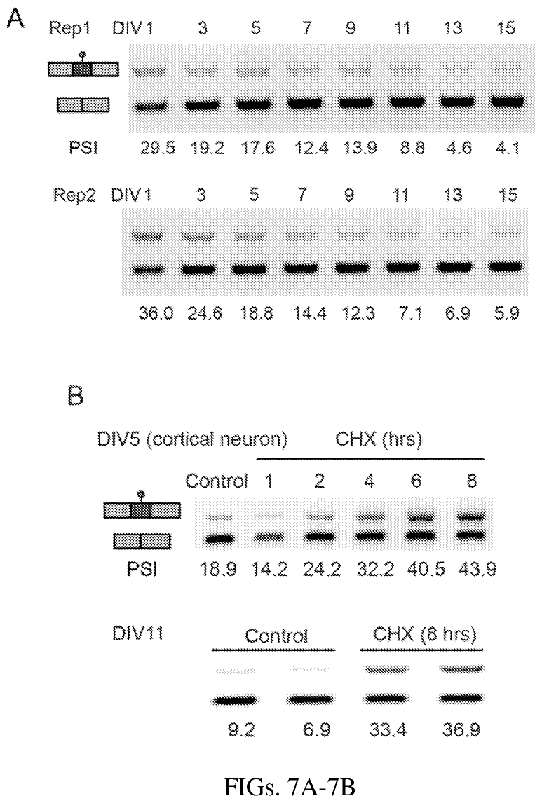

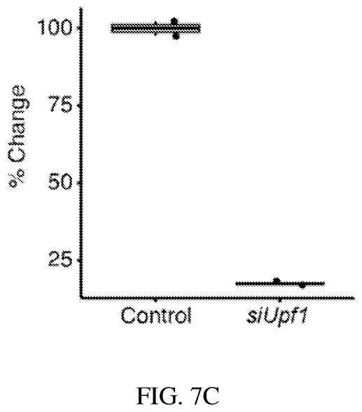

The inventors investigated cell type-specific alternative splicing during neocortical development at embryonic day 14.5 (E14.5) and identified an A3SS of Syngap1 intron10 (FIG. 1A). The inventors examined Syngap1 A3SS in mouse development and found that the inclusion level was high in non-neural tissues and during early brain development but decreased substantially in the adult brain (FIG. 1). RT-PCR of Syngap1 A3SS showed 76% inclusion at E12.5 and 5% inclusion at postnatal day 40 (P40, FIG. 1C). Notably, intronic sequences around Syngap1 A3SS are conserved in vertebrates, suggesting a selection pressure during evolution (FIG. 1B, bottom track). In cultured primary cortical neurons, Syngap1 A3SS showed 33±5% usage at day1 in vitro (DIV1) and decreased to 5±1% at DIV15 (FIG. 7A). These results suggest that the Syngap1 A3SS is included in mouse brain development and remains detectable in differentiated neurons.

Surprisingly, the Syngap1 A3SS introduces in-frame translational stop codons, which are predicted to truncate the RasGAP domain or cause NMD (FIG. 1D). Multiple lines of the results support that Syngap1 A3SS causes NMD (A3SS-NMD): 1) the predicted pre-mature stop-codons are over 50 base pairs away from downstream splice junctions (FIG. 1D); 2) an antibody against the N-terminus of Syngap1 recognized the ectopically expressed Syngap1 N-terminal fragment and the endogenous full-length protein, but was unable to detect the truncated isoform from brain lysates (FIG. 1E); 3) the Syngap1 A3SS isoform was enriched when NMD was blocked by inhibiting protein translation with cycloheximide (CHX) in Neuro2a cells and in primary neurons (FIGS. 1F and 7B); and 4) Syngap1 A3SS was upregulated when NMD was blocked by knocking down Upf1 with two different siRNAs in Neuro2a cells (FIGS. 1G and 7C). These results indicate that Syngap1 A3SS causes NMD while the canonical neuronal splice isoform ensures robust protein expression.

### Example 2: SYNGAP1 Unproductive Splicing is Functionally Conserved in Humans

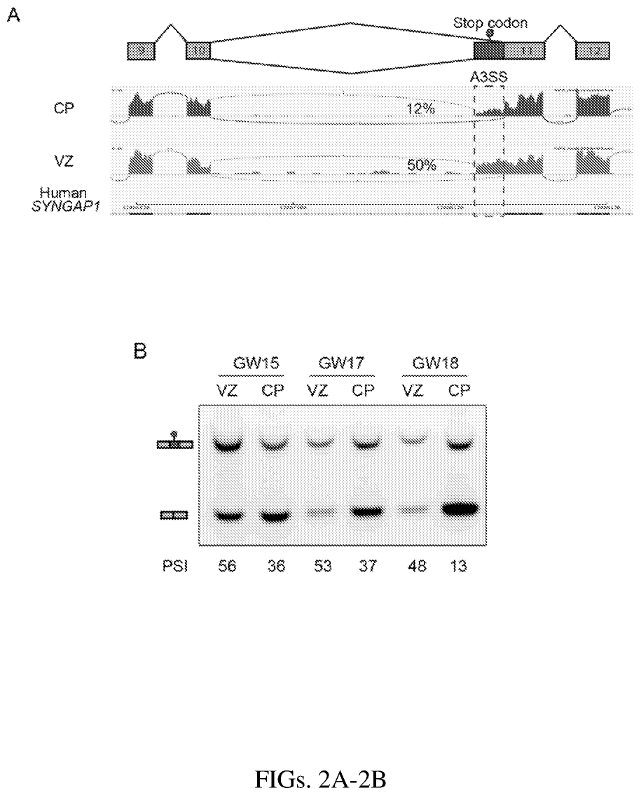

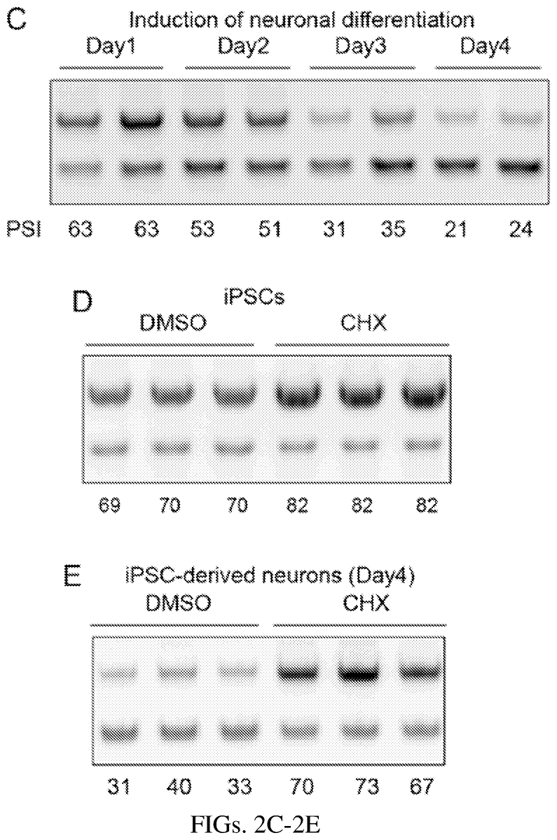

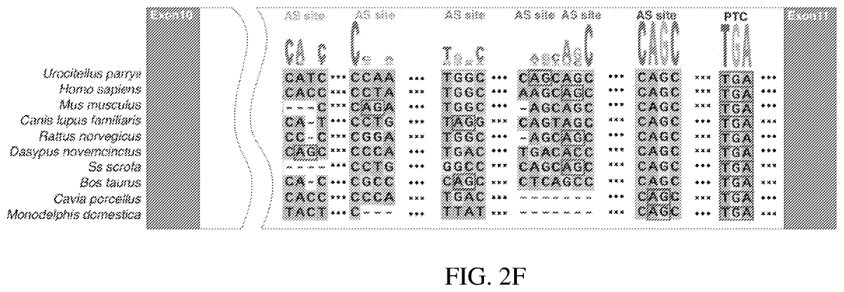

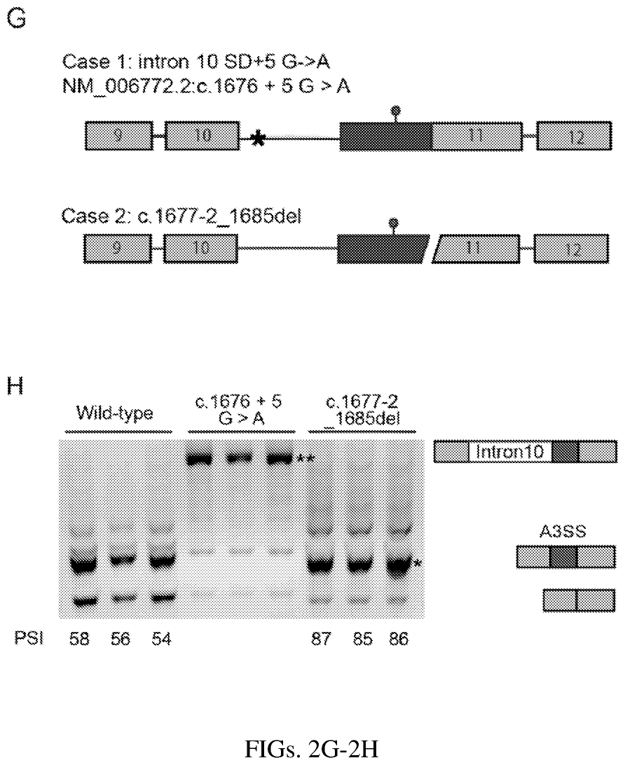

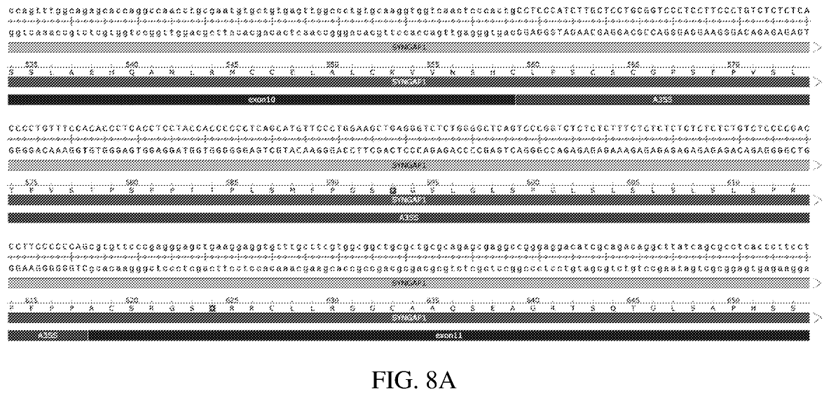

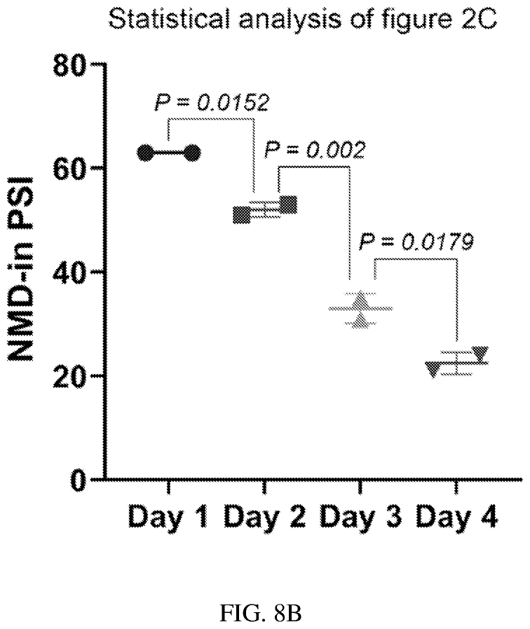

To determine whether human SYNGAP1 is regulated by A3SS in cortical development, the inventors compared RNA-Seq data between the ventricular zone (VZ, enriched for neural progenitors) and cortical plate (CP, enriched for post-mitotic neurons) of the gestational week 16 (GW16) fetal human brains (Camp et al., 2015). SYNGAP1 A3SS showed higher inclusion in the VZ than in the CP (FIG. 2A). This was further confirmed using RT-PCR with microdissected VZ and CP samples from multiple postmortem fetal dorsal forebrains (FIG. 2B). The inventors analyzed the human SYNGAP1 RNA-Seq reads and confirmed the inclusion of premature stop codons (FIG. 8A). The inventors further examined human SYNGAP1 A3SS inclusion during iPSC-to-neuron differentiation and found SYNGAP1 A3SS remained 23±2% in NGN1/2-induced neurons at day4 (FIGS. 2C and 8B). CHX treatment in iPSCs and iPSC-derived neurons significantly increased SYNGAP1 A3SS transcripts (FIGS. 2D-2E). Although the human SYGNAP1 transcript was also detectable in non-neural tissues (GTEx), the SYNGAP1 A3SS-NMD provides an orthogonal mechanism to ensure neural-specific expression of SYNGAP1 protein. Multiple sequence alignment of mammalian SYNGAP1 intron10 showed that the premature stop codon (TGA) introduced by the A3SS was highly conserved even though the positions of alternative 3′ splice sites varied among different species (FIG. 2F). These results indicate that the SYNGAP1 A3SS-NMD is functionally conserved in human neural development.

### Example 3: Human Mutations Causing SYNGAP1 Unproductive Splicing are Associated with ID and Autism

To understand whether SYNGAP1 A3SS-NMD is functional in humans, the inventors focused on two previously reported SYNGAP1 mutations in intron10: these mutations were identified in patients with typical ID and autistic features, yet the effects of these mutations on SYNGAP1 expression were not well understood (Prchalova et al., 2017; Vlaskamp et al., 2019). The inventors constructed wild-type and mutant SYNGAP1 mini-genes (spanning exon9 to exon12), introduced the constructs into Neuro2a cells, and found that the c.1676+5 G>A (NM_006772.2) mutation almost completely disrupted the splice donor and induced high intron10 retention (FIGS. 2G-2H). The pathogenic c.1677-2_1685del mutation disrupted the canonical splice acceptor of intron 10 and significantly increased SYNGAP1 A3SS usage (FIGS. 2G-2H). Thus, human mutations that lead to SYNGAP1 A3SS-NMD or intron10 retention could result in autism and intellectual disability, suggesting that the SYNGAP1 A3SS-NMD transcript is not functional in vivo.

### Example 4: SYNGAP1 A3SS-NMD is Promoted by PTBP Proteins

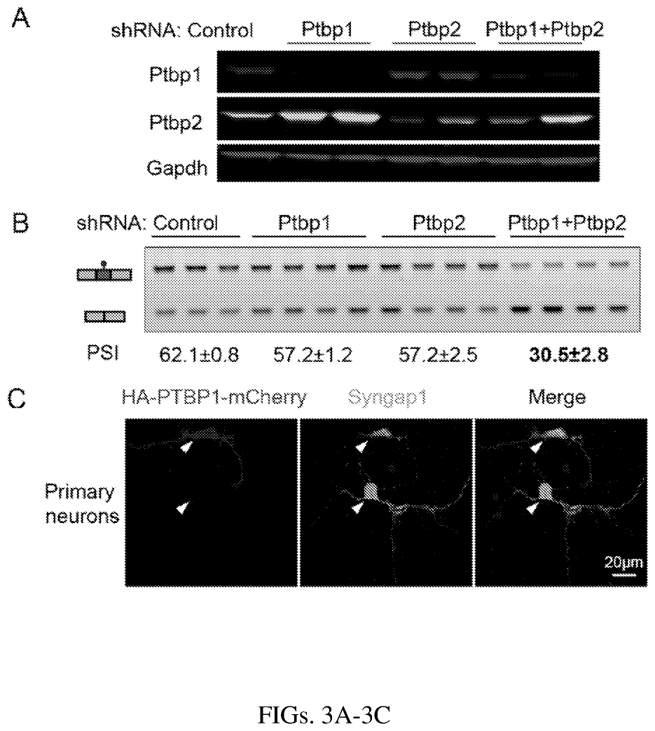

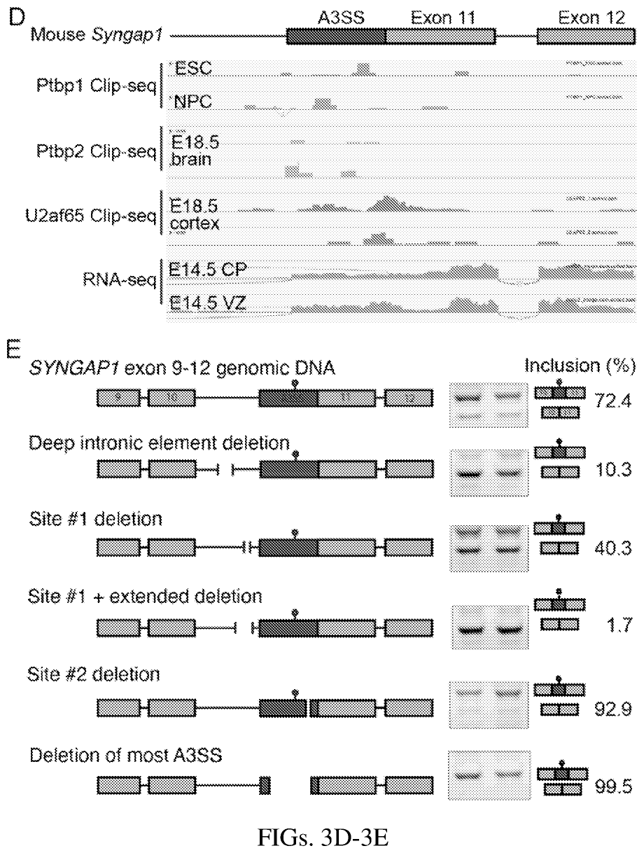

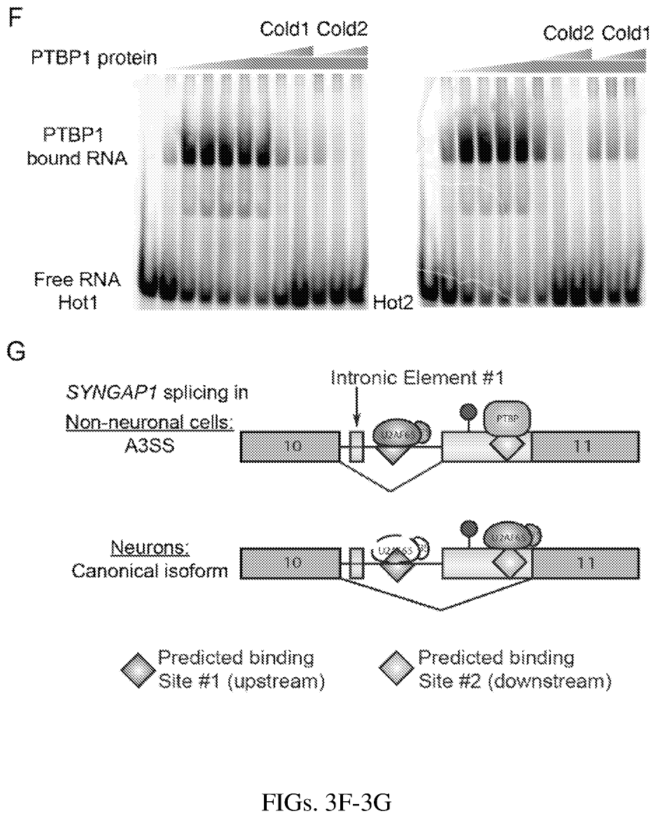

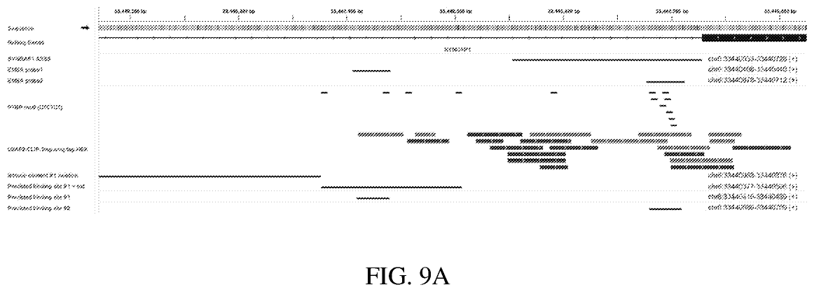

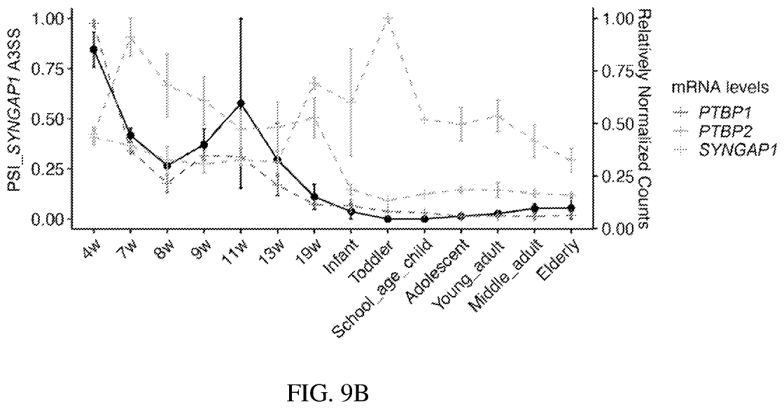

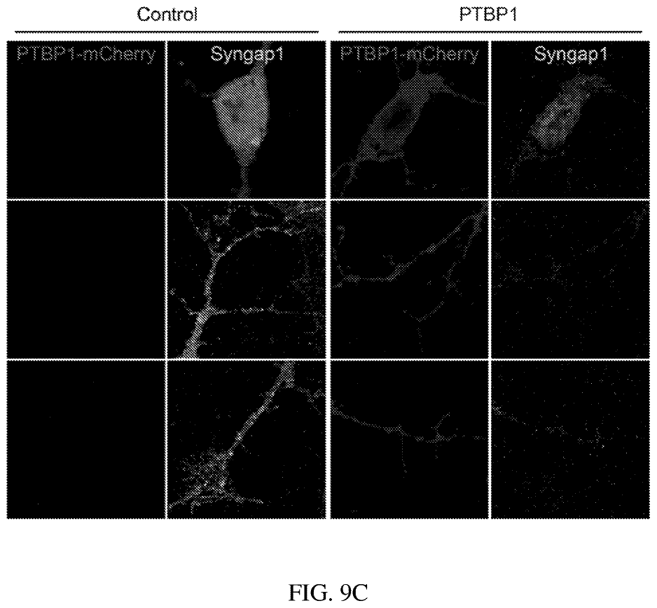

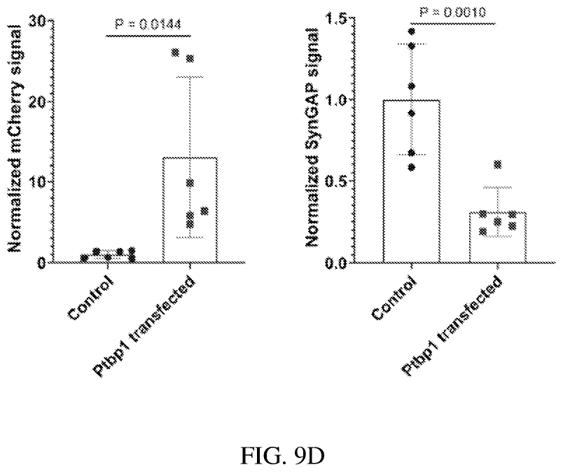

Alternative pre-mRNA splicing is regulated by RNA sequence in cis and splicing regulator proteins in trans (Darnell, 2013; Raj and Blencowe, 2015; Vuong et al., 2016). The inventors analyzed the flanking sequences of SYNGAP1 A3 SS-NMD and identified CUCUCU sequences that resemble binding motifs of PTBP1 and PTBP2 (FIG. 9A). PTBP proteins are master splicing regulators in mice and humans: PTBP1 is highly expressed in neural progenitors and non-neural tissues (GTEx), and PTBP2 is expressed in immature and differentiating neurons (Boutz et al., 2007; Vuong et al., 2016; Zhang et al., 2016). Across different developmental stages in the human dorsal forebrain (from gestational week 4 to elderly adults), SYNGAP1 A3SS-NMD inclusion ratios showed the same decreasing trend as PTBP1/2 mRNA levels (FIG. 9B). The inventors used shRNAs to knock down Ptbp1 and Ptbp2 in Neuro2a cells and found that the Syngap1 A3SS isoform was suppressed in double knockdown samples (FIGS. 3A-3B). Conversely, ectopic expression of PTBP1 in primary mouse cortical neurons decreased Syngap1 protein levels (FIGS. 3C and 9C). These results suggest that PTBP1 and PTBP2 suppress Syngap1 protein translation by promoting A3SS-NMD

To determine whether Syngap1 is a direct binding target of Ptbp proteins, the inventors re-analyzed RNA crosslinking immunoprecipitation and sequencing (CLIP-Seq) datasets of Ptbp1 in mouse ESCs and NPCs (Linares et al., 2015) and Ptbp2 CLIP-Seq results in E18.5 neocortices (Licatalosi et al., 2012). The inventors identified Ptbp1 and Ptbp2 CLIP tags in the Syngap1 intron10 between the canonical and the alternative 3′ splice sites (FIG. 3D). Ptbp proteins bind to CU-rich motifs and can redirect splicing by competing with U2af65/U2af2 (Sauliere et al., 2006), a core splicing factor that binds to the polypyrimidine tract for 3′ splice site recognition. The inventors re-analyzed CLIP-Seq data of U2af65 from E18.5 neocortices and found that CLIP-Seq tags of U2af65 and Ptbp1 overlapped with each other on Sygnap1 intron 10 (FIG. 3D), suggesting that Ptbp1/2 may compete with U2af65 and regulate the 3′ splice site.

To identify the specific sequences and potential PTBP1 binding sites required for human SYNGAP1 A3SS-NMD inclusion/skipping, the inventors created a series of mini-gene constructs in which conserved DNA sequences and predicted PTBP1 binding sites were deleted (FIG. 3E, 9A, and Methods). The inventors identified a deep intronic element and a potential U2AF65-PTBP binding site #1 (Site #1) required for SYNGAP1 A3SS inclusion. Additionally, the inventors identified a fragment within the A3SS-NMD exon that is required for the canonical/productive 3′ splice site usage of SYNGAP1 intron10. After serial deletion, the inventors narrowed it down to a 30-base pair CUCUCU-rich polypyrimidine region, designated potential U2AF65-PTBP potential binding site #2 (Site #2, FIGS. 3E and 9A). Noticeably, U2AF65/U2AF2 CLIP-Seq tags were concentrated near both splice acceptor sites in HEK293 cells (FIG. 9A).

To examine the affinity of PTBP1 to the two predicted U2AF65-PTBP binding sites #1 and #2, the inventors performed an electrophoretic mobility shift assay (EMSA) with PTBP1 protein and two sets of RNA probes (FIG. 3F). When mixed with increasing amounts of in vitro translated PTBP1 protein, Cy5-labeled probes (hot) for both Sites #1 and #2 shifted significantly; in parallel, unlabeled cold probes successfully competed with their corresponding hot probes, suggesting that PTBP1 can bind to both Sites #1 and #2 (FIG. 3F). Interestingly, cold probe #2 showed more potent competition effects to hot probe #1 than cold probe #1 (FIG. 3F, left); and consistently, cold probe #1 showed weaker competition than cold probe #2 to hot probe #2 (FIG. 3F, right). These results suggest the model that PTBP proteins bind to SYNGAP1 A3SS and compete with the splicing machinery, likely through U2AF65, for the 3′ splice site definition of intron10: in neural progenitors, PTBP1 protein is expressed, binds to U2AF65-PTBP binding site #2, and suppresses the canonical/productive SYNGAP1 splice site; whereas in neurons, PTBP proteins are turned down (FIG. 9B), resulting in exposure of the SYNGAP1 canonical splice site and increased expression of the neuronal SYNGAP1 isoform (FIG. 3G).

### Example 5: Deletion of Syngap1 A3SS-NMD in Mice Upregulates Syngap1 Protein Expression

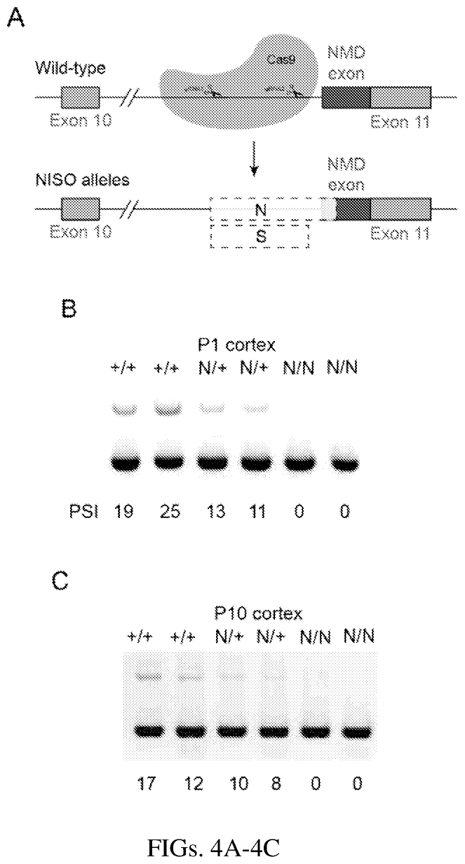

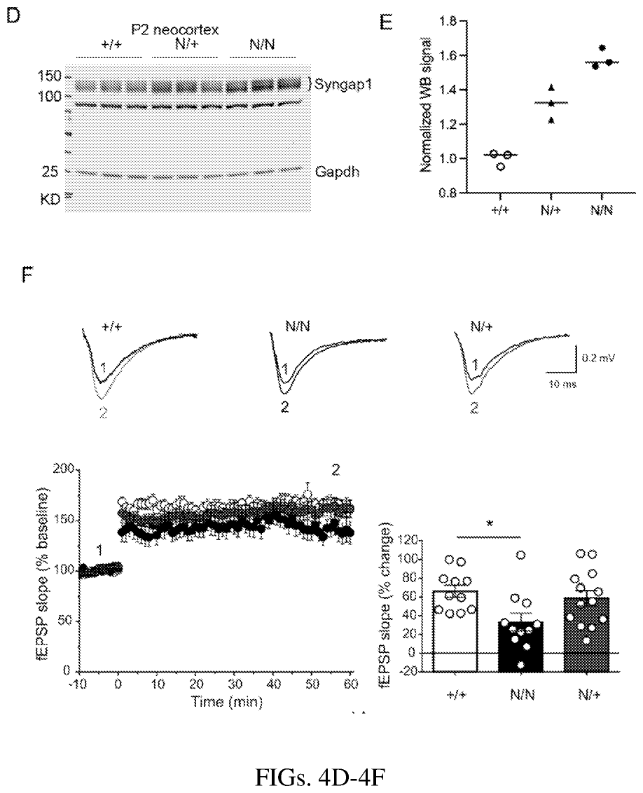

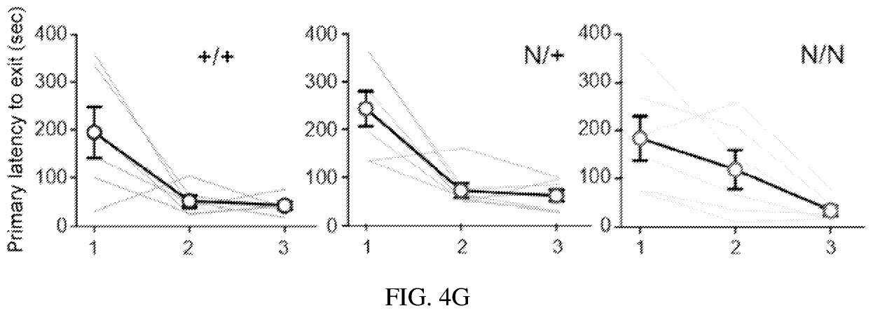

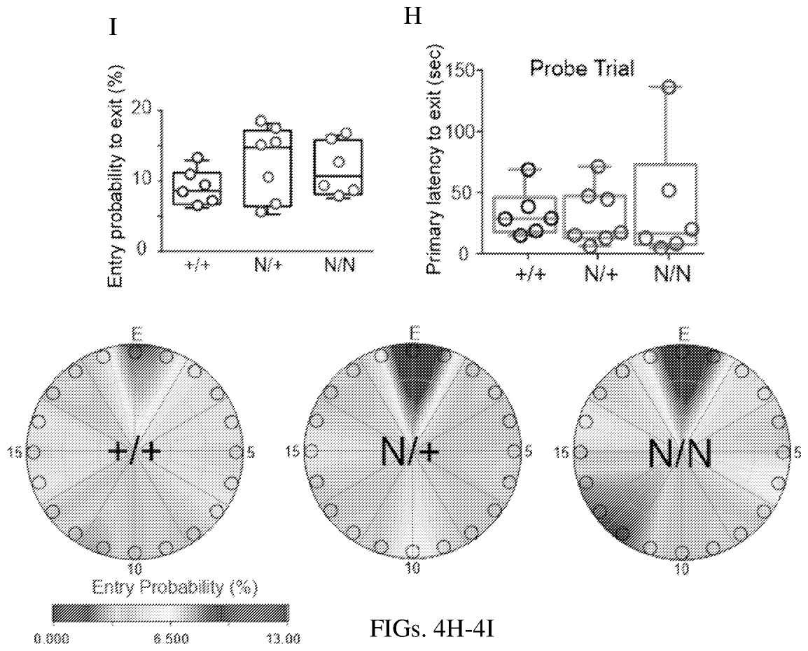

The inventors sought to understand whether Syngap1 A3SS-NMD is required for mouse development by genetically deleting this NMD event without affecting the protein-coding isoform (FIG. 4A). Based on previous findings on the deep intronic elements and predicted U2AF65 binding for A3SS-NMD inclusion (FIG. 3E), the inventors deleted these intronic elements and the A3SS with CRISPR/Cas9 and created a mouse strain named Syngap1-NISO (standing for Neuronal ISOform, 268 bp deletion, FIGS. 4A and 10A). The inventors also established a Shorter NISO allele (S allele, 160 bp deletion) where the intronic elements were deleted and the alternative 3′ splice site remained intact (FIGS. 4A and 10A). The Syngap1-NISO heterozygotes (Syngap1 N/+, and S/+) and homozygotes (Syngap1 N/N, and S/S) were born at expected ratios and appeared indistinguishable from their littermates.

The inventors confirmed the depletion of Syngap1 A3SS-NMD intron10 in Syngap1 N/N animals with RT-PCR amplification of total RNA extracted from P1 and P10 neocortices (FIGS. 4B-4C). Importantly, the inclusion of Syngap1 A3SS decreased in Syngap1-NISO heterozygotes (N/+, FIG. 4B-4C). Similarly, the Syngap1 A3SS significantly decreased in neocortices of P1 S/+ and S/S mutants (FIG. 10A-10C). The inventors focused on the N allele thereafter because it led to the complete depletion of Syngap1 A3SS-NMD in the neocortex (FIG. 4B-4C). Western blot on cortical lysates uncovered that Syngap1 protein levels were increased by 32±9% in Syngap1 N/+(adj.p<0.005) and by 58±6% in Syngap1 N/N samples at P2 (adj.p<0.001, one-way ANOVA, Dunnett's multiple comparisons) when compared with wild-type controls (+/+, FIG. 4D-4E), suggesting that one N allele can increase the protein level to 1.6 times of one wild-type allele. Syngap1 protein was also significantly increased by the Syngap1 N/+(96±22%, adj.p=0.018) and N/N (153+/−25%, adj.p=0.004) in P2 hippocampi when compared to wild-type (p<0.005, one-way ANOVA, FIG. 10D-10E). No Syngap1 protein was detected in E18.5 Syngap1 N/+ heart or lung tissues (FIG. 10G). These results indicate that suppressing Syngap1 A3SS-NMD can increase Syngap1 protein in the developing mouse brain.

Syngap1 has been shown to suppress AMPA receptor insertion at the postsynaptic membrane; Syngap1 heterozygous knockout mice showed enhanced synaptic transmission and displayed defects in learning (Clement et al., 2012). The Syngap1-NISO allele contradicts the effects of Syngap1 knockout alleles: the NISO allele increases Syngap1 protein expression, while the knockout allele decreases. Thus, the newly established Syngap1-NISO mouse model provides a unique opportunity to address whether the Syngap1 A3SS-NMD is required to express LTP and influences learning and/or memory in mice.

The inventors generated input-output curves to evaluate basal synaptic transmission and examined the paired-pulse ratio of the fEPSP to evaluate neurotransmitter release probability in adult wild-type, Syngap1 N/+ and N/N mice. Neither the input-output relationship nor the paired-pulse ratio was significantly different across the genotypes (FIG. 10I-10J). The inventors measured the LTP of field excitatory postsynaptic potentiation (fEPSP) in Syngap1 N/+ and Syngap1 N/N adult mouse hippocampi (FIG. 4F). Recordings from each of the wild-type (n=6), Syngap1 N/+(n=6), and Syngap1 N/N (n=5) genotypes showed that while LTP in wild-type and Syngap1 N/+ mutants were comparable, the magnitude of LTP was moderately but significantly reduced in Syngap1 N/N mutants (FIG. 4F). These data suggest that synaptic plasticity was affected in Syngap1 N/N but not Syngap1 N/+ adults.

The inventors next assessed performance in the Barnes maze (FIGS. 4G-4I) to determine spatial learning and memory abilities in Syngap1 N/+ and N/N mice (Pitts, 2018). Six or more adult male animals for each genotype were trained using an abbreviated Barnes maze protocol. There were three consecutive days of training followed by a fourth-day probe trial where the exit hole was closed. The primary latency to exit decreased throughout training in all genotypes (FIG. 4G). When compared to the wild-type, neither Syngap1 N/+ nor N/N mutant mice showed differences in primary latency to exit or entry probability to exit during the probe trial (FIGS. 4H-4I). The inventors also performed Rotarod assays to assess motor learning ability, and neither Syngap1 N/+ nor Syngap1 N/N animals showed a difference in the latency to fall when compared to wild-type mice (FIG. 10H). Together, these results suggest that genetic deletion of Syngap1 A3SS-NMD does not overtly impair spatial learning and memory or motor learning behaviors.

### Example 6: The NISO Allele Alleviates LTP and Membrane Excitability Deficits Caused by a Syngap1 Knockout Allele

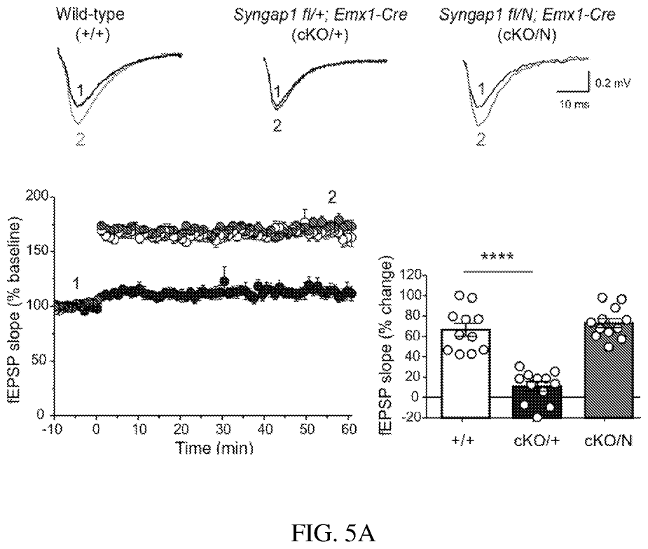

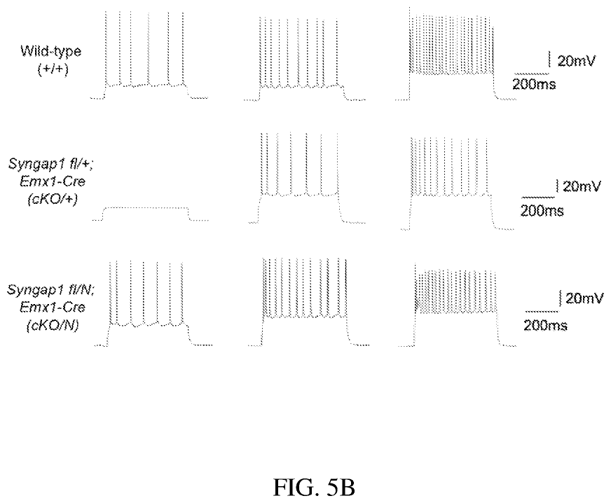

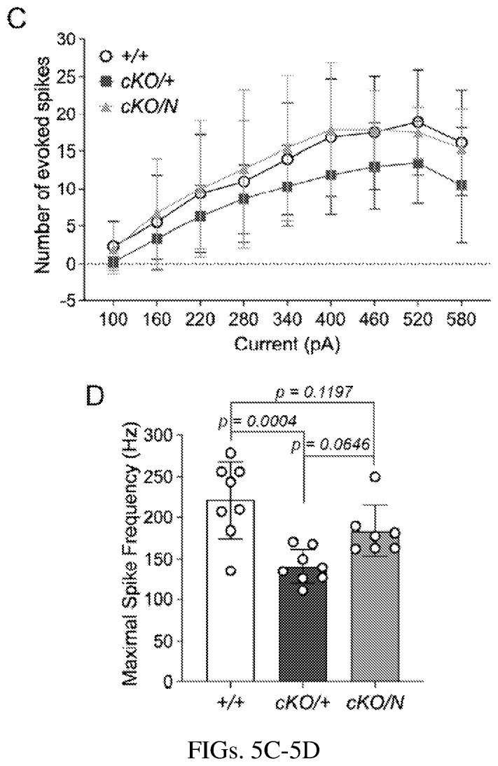

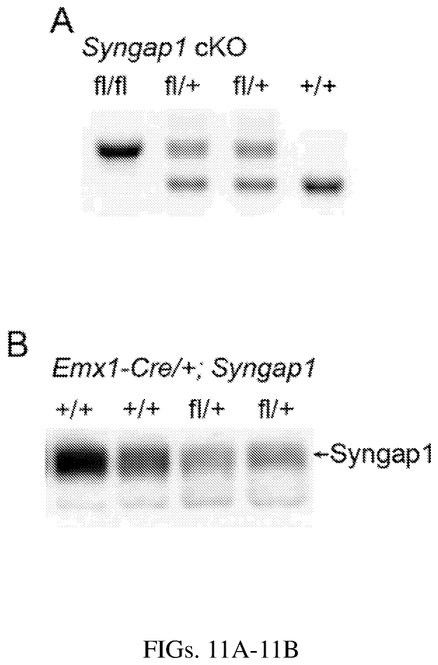

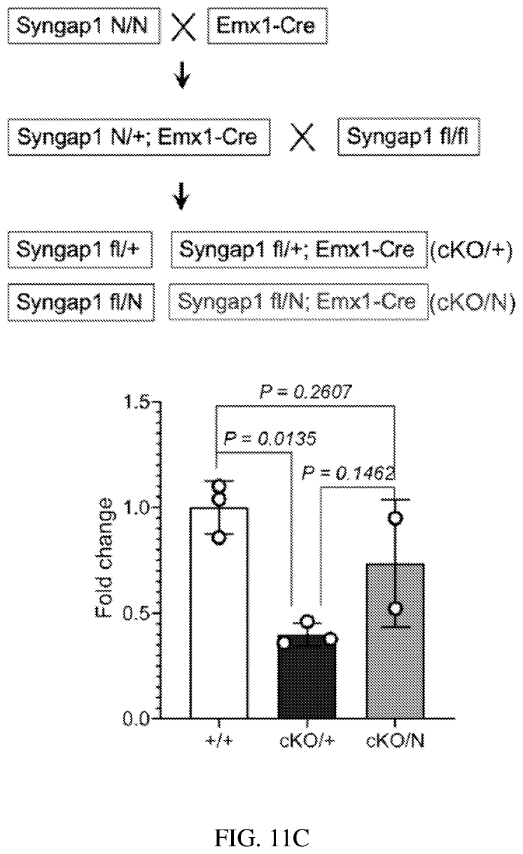

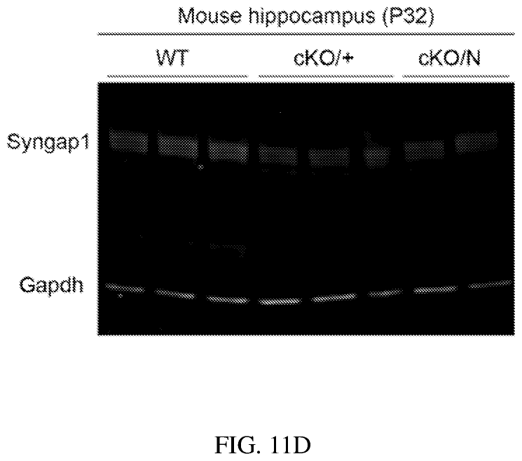

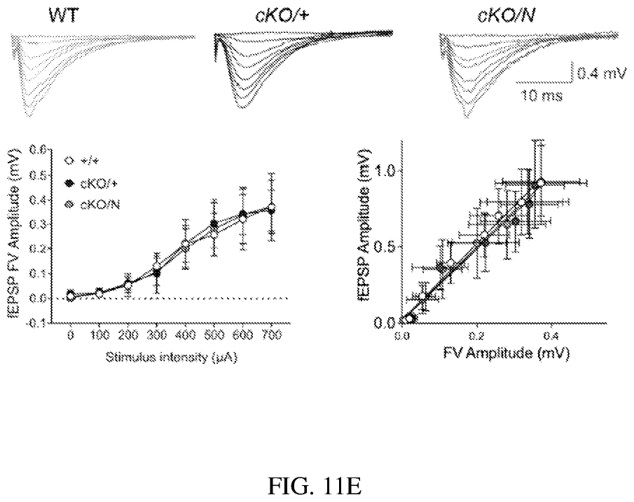

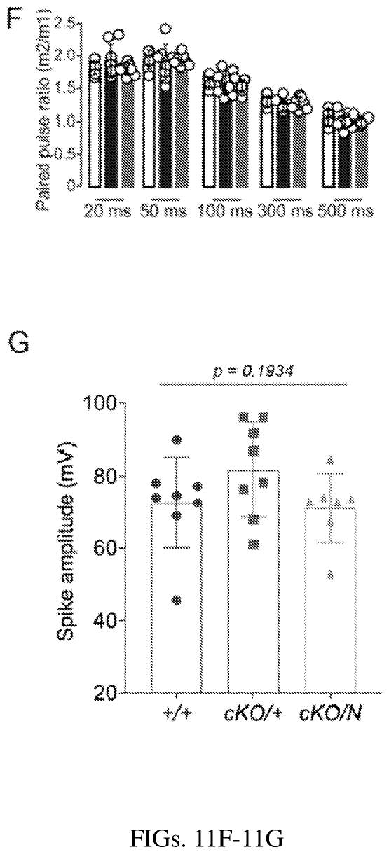

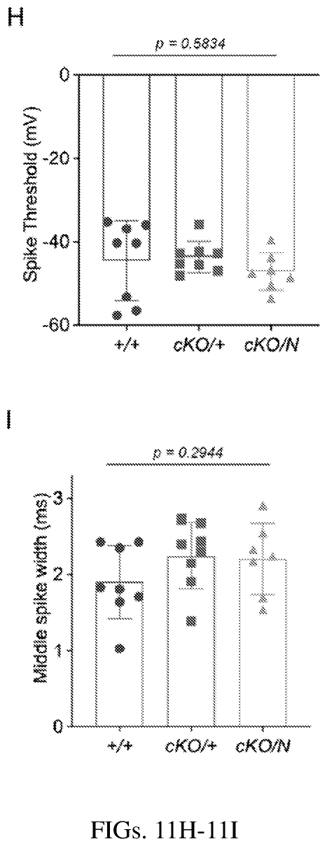

Increased Syngap1 protein by the Syngap1-NISO allele suggests that it may alleviate or rescue the Syngap1 haploinsufficiency in mice. Emx1-Cre drives Cre expression in excitatory neurons in the dorsal forebrain and is sufficient to induce Syngap1 haploinsufficient phenotypes in Syngap1 fl/+; Emx1-Cre (cKO/+) mice (Ozkan et al., 2014). The inventors validated the Syngap1 conditional knockout allele (FIG. 11A) in which Syngap1 exon6-7 were floxed (Clement et al., 2012; Komiyama et al., 2002), and confirmed that the Syngap1 protein level decreased in adult Syngap1 cKO/+ cortices (FIG. 11B). The inventors combined the Syngap1-NISO allele with the Syngap1 floxed allele and generated Syngap1 fl/N; Emx1-Cre (cKO/N) and control animals (FIG. 11C). The inventors assessed basal synaptic transmission and neurotransmitter release probability in cKO/+ and cKO/N mice and did not find a significant difference in the input-output curve or paired-pulse ratio between genotypes (FIGS. 11E-11F). The inventors then evaluated LTP of the fEPSP in the hippocampi and found the LTP was impaired in Syngap1 cKO/+ mice and rescued in Syngap1 cKO/N animals (FIG. 5A), indicating that deletion of the Syngap1 A3SS-NMD alleviated the LTP deficits caused by the Syngap1 knockout allele.

Syngap1 has been reported to maintain membrane excitability in L2/3 pyramidal neurons of mouse primary somatosensory cortex (S1)(Michaelson et al., 2018). As an independent electrophysiological test for the efficacy of the rescue strategy, the inventors performed whole-cell patch-clamp recordings on L2/3 pyramidal neurons in mouse S1 cortex. Current steps of increasing amplitude (500 ms) resulted in an increase in the number of evoked spikes (FIG. 5B-5C). Consistent with the earlier report, the inventors observed a lower number of evoked spikes in the Syngap1 cKO/+ mice (n=8) as compared to WT mice (n=8; two-way ANOVA, genotype: F2,177=8.420; p=0.0003, pairwise adj.p=0.0022). The difference in evoked spike output stayed constant over the range of injected current amplitudes (genotype×current amplitude: F16,117=0.1349, p>0.9999). In Syngap1 cKO/N mice (n=7), the excitability deficit was restored, resulting in no significant difference as compared to WT mice (adj.p=0.9306), but a significant difference as compared to the Syngap1 cKO/+ mice (adj.p=0.0011). In addition to the findings on the overall excitability, the inventors also observed that the maximal spike firing frequency (measured from the interval of the first two evoked spikes during the current injections) was lower in Syngap1 cKO/+ mice (n=8) as compared to WT mice (n=8; WT: 221.43±46.85 Hz; cKO/+: 140.73±21.04 Hz; adj.p=0.0004). In Syngap1 cKO/N mice (n=7), the higher maximal spike frequency was restored (183.99±30.95 Hz; compared to WT: adj.p=0.1197; compared to mutant: adj.p=0.0646 by one-way ANOVA, FIG. 5D). Individual action potential parameters, including overall amplitude, threshold and width at half amplitude, did not differ between the genotypes (FIG. 11G-11I). These results indicate that deletion of the Syngap1 A3SS-NMD alleviated the membrane excitability deficit caused by Syngap1 haploinsufficiency in mice.

### Example 7: SSOs Suppress SYNGAP1 A3SS-NMD in Human iPSC-Derived Neurons

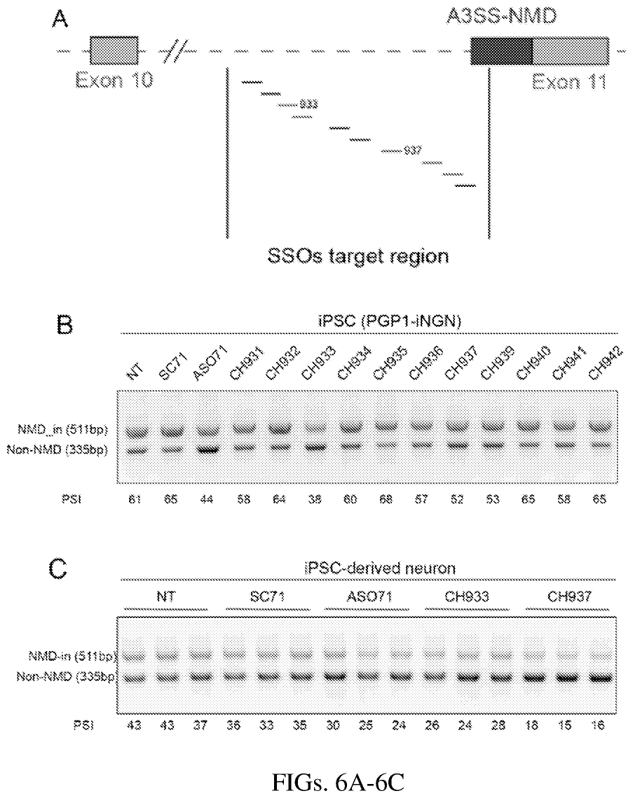

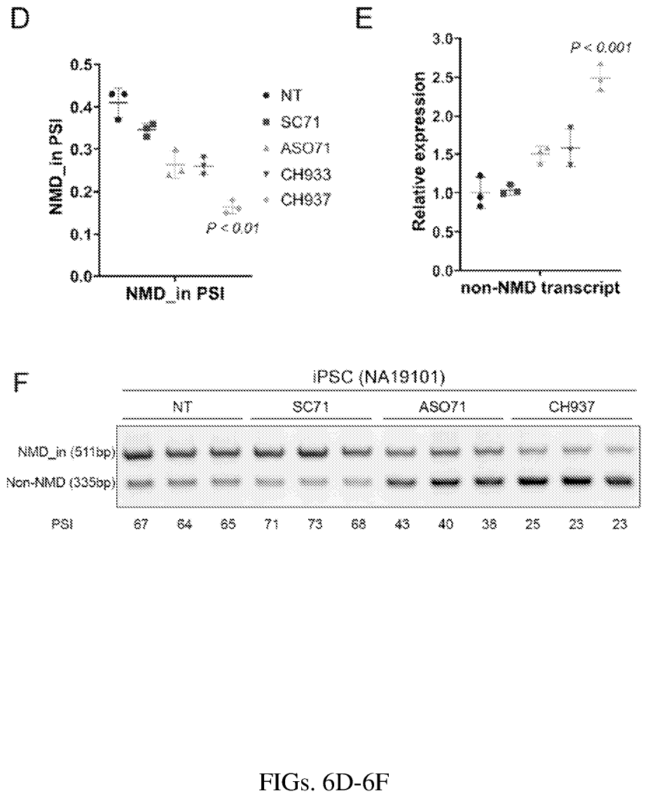

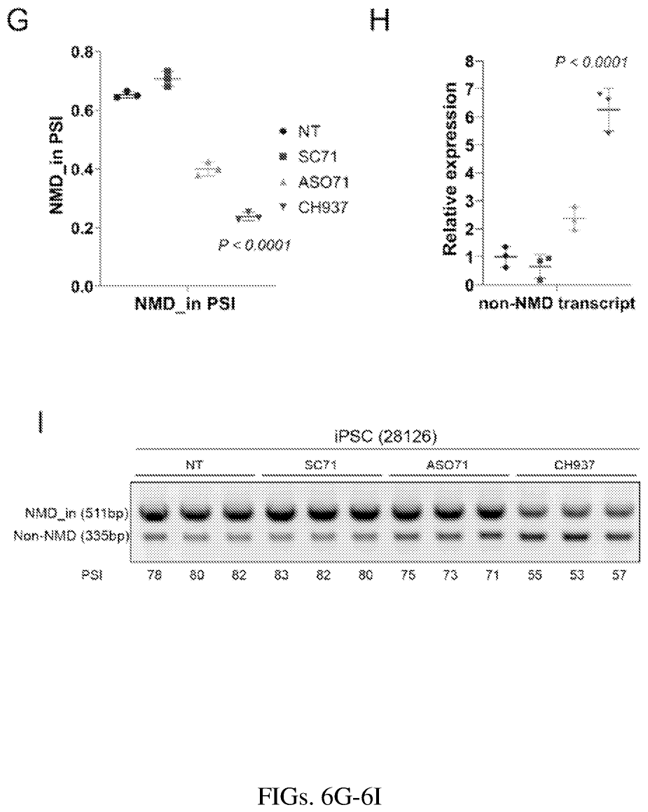

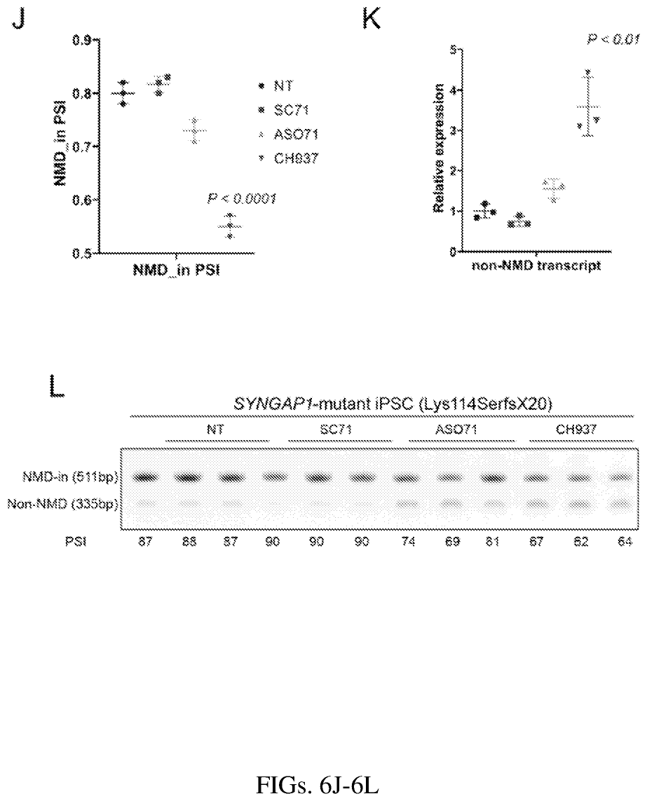

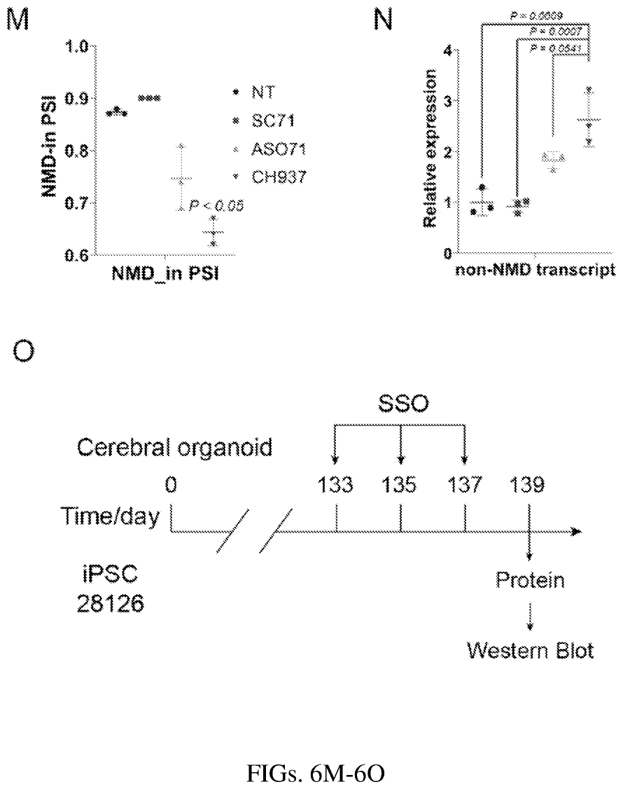

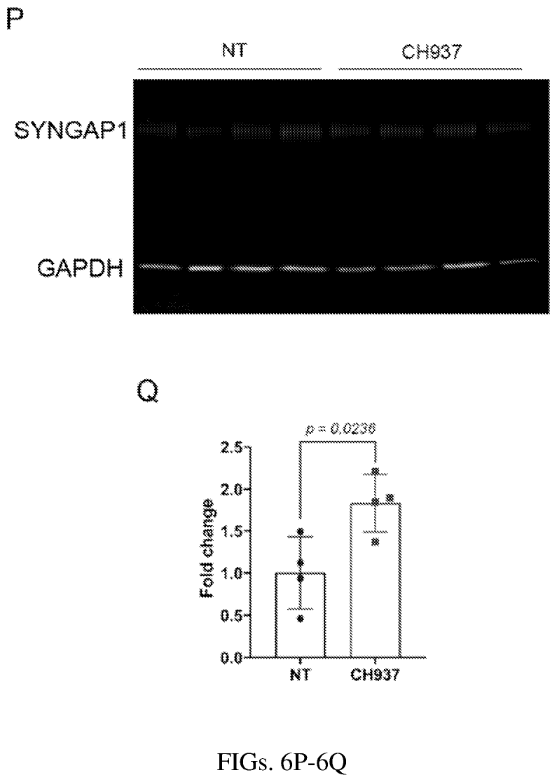

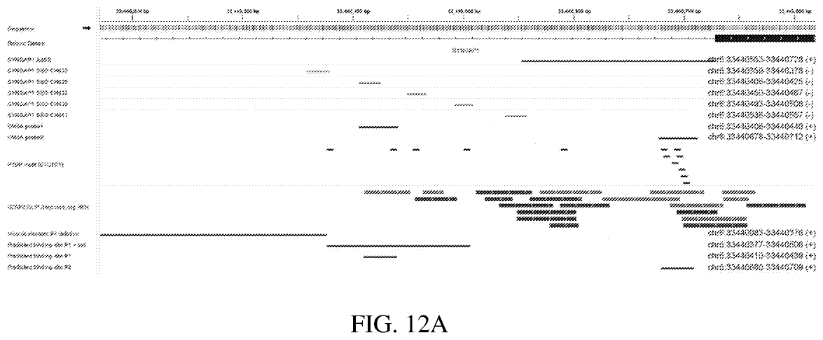

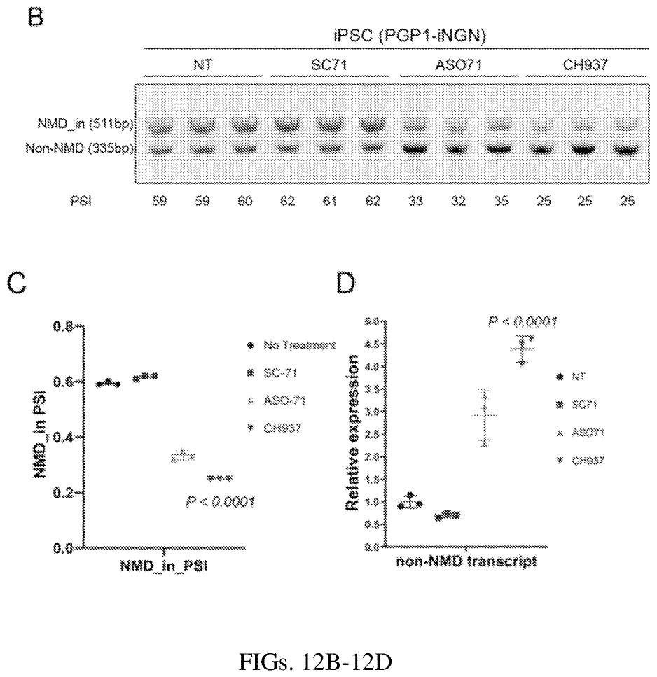

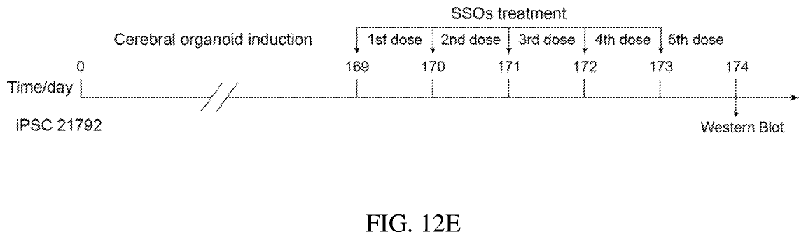

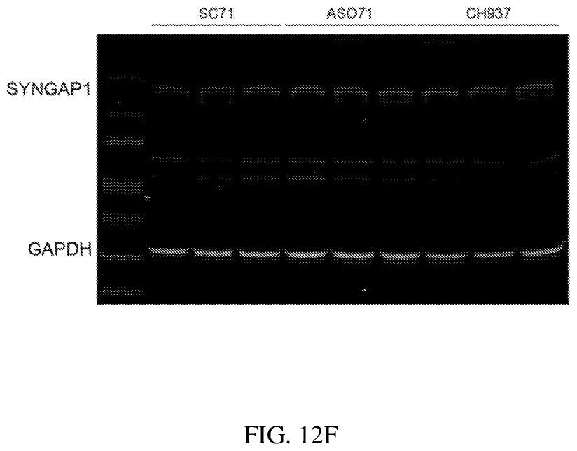

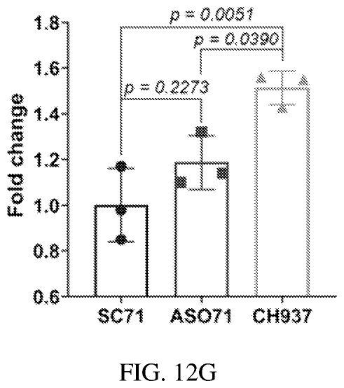

SSOs have been successfully developed to treat neurological disorders such as spinal muscular atrophy (Finkel et al., 2017), attempted for personalized medicine (Kim et al., 2019), and suggested to treat haploinsufficient human diseases (Han et al., 2020). Increased Syngap1 protein levels due to Syngap1 A3SS deletion in mice and the conserved SYNGAP1 A3SS in human brains led us to determine whether the SYNGAP1 productive isoform can be upregulated in human cells through SSO-mediated suppression of SYNGAP1 A3SS-NMD. SSOs with (2′-MOE) chemistry have been successfully developed to redirect splicing, manipulate gene expression, and treat diseases (Kole et al., 2012). To identify SSOs that suppress human SYNGAP1 A3SS-NMD, the inventors performed a rationale design by targeting the following regions/sequences (FIG. 6A): 1) critical splice elements identified through serial deletion of the human SYNGAP1 mini-gene (FIG. 3); 2) predicted splicing regulatory sequences; 3) predicted stem-loop structures and conserved sequences that overlap with experimentally identified splice elements (FIGS. 6A and 12A).

The inventors synthesized eleven SSOs using a phosphorothioate backbone with 2′-MOE modified residues and tested them in human iPSCs (FIGS. 6A-6B). SSOs CH933 and CH937 most efficiently suppressed A3SS-NMD inclusion in a human iPSC line (PGP1-iNGN, FIGS. 6B and 12A-12C). The inventors delivered SSOs CH933 and CH937, a scrambled control, and a previously reported ASO71 (Lim et al., 2020) into human iPSC-derived neurons and found that CH937 was the most effective in decreasing the A3SS-NMD inclusion (FIGS. 6C-6D). The inventors further measured the productive/functional SYNGAP1 transcript using Q-PCR primers specific to the non-NMD isoform and CH937 induced a 2.5-fold increase of functional SYNGAP1 mRNA (Tukey's multiple comparisons test, adj.p<0.001, FIG. 6E). In contrast, the ASO71 developed in HEK293 cells was less efficient in upregulating SYNGAP1 transcript in human iPSC-derived neurons (1.5 fold, adj.p=0.029, FIG. 6E). These results indicate that the lead SSO CH937 can effectively suppress human A3SS-NMD and increase the productive SYNGAP1 isoform in human iPSCs and iPSC-derived neurons.

The inventors further examined the effects of the SSO CH937 in two additional control human iPSC lines (NA19101 and 28126) and confirmed that CH937 was more effective than ASO71 in decreasing the SYNGAP1 A3SS-NMD inclusion (FIGS. 6F-6K). Importantly, CH937 significantly increased the functional SYNGAP1 transcript to 6.3-fold and 3.6-fold of non-treated controls in NA19101 and 28126, respectively (FIGS. 6H, 6K). Next, the inventors delivered CH937 to a SYNGAP1 patient-derived iPSC line harboring a heterozygous frame-shift mutation (Lys114SerfsX20) and found that the SSO CH937 decreased SYNGAP1 A3SS-NMD inclusion (FIGS. 6L-6M) and significantly increased functional SYNGAP1 transcript to 2.6-fold of non-treated controls (FIG. 6N). These results support that the lead SSO CH937 can effectively suppress SYNGAP1 A3SS-NMD and increase functional SYNGAP1 isoform levels in SYNGAP1 patient-derived iPSCs.

To determine whether suppressing SYNGAP1 A3SS-NMD can upregulate SYNGAP1 protein, the inventors determined the effects of SSO CH937 in iPSC-derived brain organoids. Cerebral organoids were induced from human iPSCs (28126) following an established protocol (Yoon et al., 2019), transfected with CH937 at post-induction days 133, 135, and 137, and harvested at day 139 for protein analysis (FIGS. 6O-6P). CH937 significantly increased SYNGAP1 protein when compared with control organoids (83%±28%, p<0.05 by unpaired t-test). (FIG. 6Q) The inventors further tested SSO CH937 in cerebral organoids derived from another iPSC line (21792) at post-induction days 174, and again observed significantly increased SYNGAP1 protein when compared with control oligonucleotides (FIG. 12E-12G, increased by 51%±10%, adj.p=0.0051 by one-way ANOVA). These results suggest that the SSO CH937 can upregulate SYNGAP1 protein in human iPSC-derived cerebral organoids.

### Example 8: Methods and Materials

### Molecular Cloning

To detect the A3SS with RT-PCR, the human SYNGAP1 alternative exon11 was amplified with primer pairs CH748-CH749, and the mouse Syngap1 A3SS was amplified with primer pairs CH743-CH744.

pCAG-SYNGAP1(N-terminus): the N-terminal SYNGAP1 coding sequence was amplified using SG020 and SG022 and inserted into pCZ01 using Gibson Assembly (NEB). pCZ01 is a modified pCAG-IG vector described previously (Zhang et al., 2016).

pCAG-HA-Flag-PTBP1-IRES-EGFP: PTBP1cDNA was amplified with CH448-CH422-2, digested with AscI and NotI, and ligated into linearized pCZ01.

For in vitro translation, PTBP1 cDNA was amplified using primer pair SG209-SG210. To knock down Ptbp1/2, shRNA lenti-vectors used as described previously (Zhang et al., 2016).

The SYNGAP1 wild-type mini-gene construct spanning exon9 through exon12: genomic DNA extracted from HEK293FT cells was amplified with primer pairs SG085-SG086 and the purified PCR product was inserted to pCZ01 using Gibson Assembly (NEB).

### SYNGAP1 Mini-Gene Deletion Constructs:

To delete the deep intronic element, two fragments were amplified using PCR primer pairs SG159-SG086 and SG160-SG085, and inserted into linearized pCZ01.

To delete predicted U2AF65 binding site #1, two fragments were amplified using PCR primer pairs SG155-SG086 and SG156-SG085, and inserted into pCZ01.

To delete predicted U2AF65 binding site #1+ extended, two fragments were amplified using PCR primer pairs SG095-SG086 and SG098-SG085, and inserted into pCZ01.

To delete the A3SS, two fragments were amplified using PCR primer pairs SG087-SG086 and SG088-SG085, and inserted into pCZ01.

To delete predicted U2AF65 binding site #2, two fragments were amplified using PCR primer pairs SG089-SG086 and SG090-SG085, and inserted into pCZ01 using Gibson Assembly.

SYNGAP1 Mini-Gene with Patient Mutations:

To introduce the mutation c.1676+5 G>A (NM_006772.2), two fragments were amplified using PCR primer pairs SG189-SG086 and SG190-SG085, and inserted into pCZ01 using Gibson Assembly.

To introduce the mutation c.1677-2_1685del, two fragments were amplified using PCR primer pairs SG191-SG086 and SG192-SG085, and inserted into pCZ01 using Gibson Assembly.

Plasmids were transfected into Neuro2a cells (ATCC) with Lipofectamine 2000 (Thermo Fisher), selected by puromycin and total RNA was extracted with Trizol (Sigma). Reverse transcription was performed with random primers following manufacturer's protocols (Superscript IV, Thermo Fisher).

### EMSA

Cy5 conjugated RNA probes (SG-probe1/2) and unlabeled cold competitors of potential PTBP1 binding sites were synthesized by IDT. PTBP1 protein was produced by TnT SP6 High-Yield Wheat Germ Protein Expression System (Promega). In vitro translated PTBP1 was diluted in the RNA-protein binding solution, incubated with Cy5 probes with or without cold competitor probes, and resolved on 8% TBE gels (Thermo Fisher, EC6215BOX). The gel was directly visualized with a Typhoon imaging system.

### Primary Neuron Culture and Immunostaining

Primary hippocampal neurons (from E18.5 CD1 mouse embryos or neonatal pups) or cortical neurons (from E15.5 embryos) were isolated with Papain (Worthington) and cultured in vitro. Primary neurons were plated onto poly D-lysine coated coverslips and cultured following standard protocols (Neurobasal medium supplemented with GlutaMax, N2, B27, and 1 μM AraC during DIV1-DIV3). Primary neurons were transfected by Lipofectamine 2000 (Thermo Fisher) or jetOPTIMUS (Polyplus). For immunostaining, primary neurons were fixed 10 minutes in 4% paraformaldehyde (PFA) at 4° C., rinsed with 1×PBS, and incubated with blocking buffer (1×PBS containing 0.03% Triton X-100 and 5% normal donkey serum) in room temperature for 30 mins, and further incubated with primary antibodies diluted in PBST buffer (1×PBS containing 0.03% Triton X-100) overnight at 4° C. After 3 times washing with 1×PBS, slides were incubated for one hour at room temperature with fluorophore-conjugated secondary antibodies in the dark. Slides were scanned with a Leica SP5 confocal or Zeiss Apotome 2 microscope. The antibodies are listed in Table 1.

### Mouse Protocols

Mouse protocols were reviewed and approved by the University of Chicago Institutional Animal Care and Use Committee. Guide RNAs (SG321 and SH322) flanking the designed Syngap1 deletion region were selected with the CRIPSOR online tool. Guide RNAs, tracrRNA, and Cas9 protein were purchased from IDT. Guide RNAs were annealed with tracrRNA, mixed with Cas9 protein in the injection buffer, and injected into C57BL/6 mouse zygotes by the Transgenic Core (U Chicago). Founder mice were PCR screened for the deletion and positive founders were bred with C57BL/6 (Charles River) to obtain positive F is, which were further mated with C57BL/6 for positive F2s. F2s and later generations were used in this study. The Syngap1-NISO allele is genotyped using primers SG245F-SG331R-SG202R, and the expected product sizes are: wild-type allele is 405 bp (and weak 761 bp), and the NISO N allele is 493 bp. The Syngap1 conditional knockout (cKO, Jax #029303) and Emx1-Cre (Jax #005628) mice were obtained from the Jackson Lab and genotyped following providers' protocols.

Barnes maze test was performed following published protocols (Arias-Cavieres et al., 2020; Pitts, 2018). Motor coordination and balance of mice (aged around 2 months to 4 months) were examined by Rotarod tests: mice were gently placed on a rotatable rod (Columbus Instruments, ECONOMEX) that was accelerated at 0.2 rpm/second from 5 rpm over a 3-minute period. Time from rotation start to mouse fall in each trial was recorded as the latency to fall. Three trials were performed for each mouse on the test day.

### Electrophysiology

Acute hippocampal slices were prepared from young adult (1-2.5 months) male mice, which were anesthetized with isoflurane and euthanized by rapid decapitation. The brain was rapidly harvested and blocked, rinsed with cold artificial cerebrospinal fluid (aCSF) and mounted for vibratome sectioning. The mounted brain tissue was submerged in aCSF (4° C.; equilibrated with 95% O₂, 5% CO₂) and coronal cortico-hippocampal brain slices (350 μm thick) were prepared. Slices were immediately transferred into a holding chamber containing aCSF equilibrated with 95% O₂, 5% CO₂ (at 20.5±1° C.). Slices were allowed to recover a minimum of one hour prior to the transfer into recording chamber and were used within eight hours following tissue harvest. The composition of aCSF (in mM):118 NaCl, 10 Glucose, 20 sucrose, 25 NaHCO₃, 3.0 KCl, 1.5 CaCl₂, 1.0 NaH₂PO₄ and 1.0 MgCl₂. The osmolarity of aCSF was 305-315 mOsm and equilibrated, and the pH was 7.42±0.02.

The extracellular recording of the field excitatory postsynaptic potential (fEPSP) was established in aCSF (31.0±2° C., equilibrated with 95% O₂ 5% CO₂) superfused and recirculated over the preparation. The stimulation electrode, a custom constructed bipolar electrode composed of twisted Teflon coated platinum wires (wire diameter: 127 μm, catalog number 778000, AM Systems.), was positioned in the Schaffer Collateral and the recording electrode (1-2 MΩ) was placed into the stratum radiatum of the CA1. The intensity of the electrical current (100-400 μA; 0.1-0.2 ms duration) was set to the minimum intensity required to generate the 50% maximal fEPSP. After 10 minutes of recording the baseline fEPSP, LTP was induced using Theta Burst Stimulation (TBS: four trains of 10 bursts at 5 Hz, each burst was comprised four pulses at 100 Hz). Following stimulation, recordings continued for up to one hour. The fEPSP slope was normalized to baseline values. Recordings were made using either a Multiclamp 700B (Molecular Devices, San Jose, CA, USA) or using a differential amplifier (AM system, Washington, DC, USA).

Whole-Cell Patch-Clamp Recordings from Neocortical Pyramidal Neurons

Slices from primary somatosensory cortex (S1; 350 μm thick) were prepared from young mice (postnatal day 16-28) after isoflurane anesthesia and decapitation. The procedure was described in detail before (Gill and Hansel, 2020) and is in accordance with the guidelines of the Animal Care and Use Committee of the University of Chicago. The slices were cut on a vibratome (Leica VT1000S) using ceramic blades. To preserve thalamocortical projections, slices were cut at a 550 angle to the right (posterior) of the anterior-to-posterior axis of the brain (see Agmon and Connors, 1991). The slices were cut in a sucrose slicing solution containing the following (in mM): 185 sucrose, 2.5 KCl, 25 glucose, 25 NaHCO₃, 1.2 NaH₂PO₄, 0.5 CaCl₂, and 0.5 MgCl₂, bubbled with 95% O₂ and 5% CO₂. Following slicing, the slices were kept in artificial cerebrospinal fluid (ACSF) containing the following (in mM): 124 NaCl, 5 KCl, 1.25 NaH₂PO₄, 2 CaCl₂, 2 MgSO₄, 26 NaHCO₃, and 10 D-glucose, bubbled with 95% O₂ and 5% CO₂. The slices were allowed to recover for at least 1 h and were subsequently transferred to a submerged recording chamber superfused with ACSF at elevated temperature (28-30° C.). Whole-cell patch-clamp recordings were performed under visual control using a 40× water-immersion objective in combination with near-infrared light illumination (IR-DIC) and a Zeiss AxioCam MRm camera mounted on a Zeiss Examiner A1 microscope (Carl Zeiss MicroImaging). Patch pipettes (~2.5-4.5 MΩ) were filled with internal saline containing the following (in mM): 9 KCl, 10 KOH, 120 K-gluconate, 3.48 MgCl₂, 10 HEPES, 4 NaCl, 4 Na₂ATP, 0.4 Na₃GTP, and 17.5 sucrose, pH adjusted to 7.25. Patch-clamp recordings were performed in current-clamp mode using an EPC-10 amplifier (HEKA Electronics). Input resistance (Ri) was measured by injection of hyperpolarizing test currents (100 pA, 100 ms). For all recordings and analyses, the inventors used a blind approach, in which the researcher was ignorant of the mouse genotype. Data obtained from the patch-clamp recordings were analyzed using Pulsefit (HEKA Electronics), Igor Pro (WaveMetrics) and MatLab. For statistical comparison of action potential-related parameters between genotypes, the inventors used the one-way ANOVA. A two-way ANOVA test was used to examine statistical relationships between a) genotypes and b) genotypes×current amplitude in the input (current injection)−output (spike number) measurements.

Human Induced Pluripotent Stem Cells (iPSCs)

iPSC lines, including PGP1-iNGN (Busskamp et al., 2014), SYNGAP1 (Lys114SerfsX20, Simons Foundation), 28126, 21792, and NA19101 (Gilad lab at UChicago), were grown in Essential 8 (Thermo Fisher, A1517001) with penicillin-streptomycin (100 U/mL, Thermo Fisher, 15140122) in 10-cm dishes coated with GelTrex (Thermo Fisher, A1413301) in an incubator at 37° C. with 5% CO2. Instead of Essential 8, SYNGAP1-mutant iPSCs were grown in StemFlex (Thermo Fisher, A3349401). Rock inhibitor Y-27632 dihydrochloride (10 μM, Tocris, 1254) was added into culture media for 24 hours after passage. For neuron induction, PGP1-iNGN iPSCs were grown in Essential 8 supplemented with doxycycline (1 μg/mL, Sigma-Aldrich, D9891) and penicillin-streptomycin (100 U/mL) for 4 days. To analyze the NMD-transcript of SYNGAP1, iPSCs cells and iPSC-derived neurons were treated with cycloheximide (200 μg/mL dissolved in DMSO) for 5 hours in 12-well plates with 1 mL culture media in each well, followed by RNA extraction and analyses.

### Cerebral Organoid Culture

Induction of brain organoids was performed according to published protocols (Yoon et al., 2019). Briefly, 3×10⁶ hiPSCs were seeded per AggreWell 800 well (STEMCELL Technologies, 34815) in Essential 8 supplemented with Y-27632 dihydrochloride (10 μM) (Tocris, 1254) and penicillin-streptomycin (100 U/mL). 24 hours later, iPSC spheroids were transferred into ultra-low attachment 6-well plates and then incubated in Essential 6 supplemented with dorsomorphin (2.5 μM) (Sigma-Aldrich, P5499) and SB-431542 (10 PM) (Tocris, 1614) for 6 days to induce neural spheroids. Then the neural spheroids were incubated in Neurobasal A medium (Thermo Fisher Scientific, 10888022) supplemented with B-27 without vitamin A (1:50) (Thermo Fisher Scientific, 12587010), GlutaMax (1:100) (Thermo Fisher Scientific, 35050-061), epidermal growth factor (EGF) (20 ng/mL) (R&D Systems, 236-EG), basic fibroblast growth factor (bFGF) (20 ng/mL) (R&D Systems, 233-FB) and penicillin-streptomycin (100 U/mL) for 19 days, after which the EGF and bFGF were replaced by brain-derived neurotrophic factor (BDNF) (20 ng/mL) and NT-3 (20 ng/mL) for 18 days. From the 43^{rd} day, brain organoids were incubated in Neurobasal A media supplemented with B-27 without vitamin A (1:50), GlutaMax (1:100) and penicillin-streptomycin (100 U/mL) for long-term culture.

### Splice Switching Oligonucleotides (SSOs)

SSOs were synthesized by Integrated DNA Technologies (IDT). For SSO transfection, 4×10⁵ cells were seeded in 12-well plates coated with GelTrex (Thermo Fisher, Gibco, A1413301). 200 nM SSOs were transfected into cells using TransIT®-LT1 Transfection Reagent (Mirus, MIR2300) according to the manufacturer's instructions. Brain organoids derived from iPSC 28126 were treated by three doses of SSOs (300 nM) from day 133 to 137. On day 139, RNA and protein were extracted from brain organoids for PCR and western blot analyses. For brain organoids derived from iPSC 21792, they were treated by five doses of ASOs (200 nM) from day 169 to 173. RNA and protein were extracted on day 174. Total RNA was extracted using TRIzol reagent (Thermo Fisher, 15596018) and Direct-zol RNA Purification Kit (Zymo Research, R2060) 24 hours after transfection. cDNA was synthesized by SuperScript IV Reverse Transcriptase kit (Thermo Fisher, 18090050). Quantitative PCR (Q-PCR) was performed using SYBR Green PCR Master Mix (Thermo Fisher, 4344463) in QuanStudio Real-Time PCR Systems (Thermo Fisher, ZG11CQS3STD) according to manufacturers' instructions.

### RT-PCR and Western Blot

For RNA extraction, brain tissues, or culture cells were dissolved in TRIzol by firmly pipetting and then subjected to either precipitation or Direct-zol RNA Purification Kit. For Western blotting, protein lysates were extracted with RIPA buffer (Thermo Fisher, PI89901) supplemented with proteinase inhibitors (Sigma-Aldrich, 11836170001). Protein samples were loaded onto SDS-PAGE gels, transferred to PVDF membranes, incubated with primary and secondary antibodies successively (Table 1), and then imaged using the LI-COR Odyssey system (LI-COR, 9142).

### Data Analyses and Statistical Testing

To identify differential splicing events, the inventors analyzed at least two biological replicates for each genotype using rMATS and filtered by False Discovery Rate (FDR)<0.05 and |differential PSI|>10%. CLIP-Seq data were aligned and mapped to hg38 genome by CLIPSeqTools, using the default parameters. The aligned files were visualized in IGV. CLIP-Seq datasets used in this study: PTBP1_ESC (SRR2121761), PTBP1_NPC (SRR2121762), U2AF65 (ERR208893, ERR208897, GSE83923), PTBP2 (SRR871026, SRR871030). Multiple Sequence

Alignment Genome sequences of multiple vertebrates were obtained from NCBI HomoloGene database. Multiple sequence alignment and phylogeny analysis were performed by MAFFT over the conserved region of SYNGAP1 intron10. The splice site usage was validated by deposited RNA-Seq data of non-neuronal tissues from the corresponding species. Comparisons of electrophysiology recordings were assessed by a one-way ANOVA followed by the Bonferroni correction in Prism (GraphPad). Comparisons of mouse behaviors and Q-PCR results were done with Tukey's multiple comparisons in Prism, and the adjusted p-values were indicated.

### List of Reagents and Tools

Reagents and tools useful for practicing certain aspects described herein are listed in Table 1.

TABLE 1

| Reagent/Resource | Source | Identifier |
|---|---|---|
| Anti-Syngap1 (C terminal) | Thermo-Fisher | Cat# PA1-046 |
| Anti-Syngap1 (N terminal) | Thermo-Fisher | Cat# PA5-37909 |
| Anti-HA | Roche | 12158167001 |
| Anti-Ptbp1 | Abcam | Cat# ab5642 |
| Anti-Ptbp2 | Millipore | Cat# ABE431 |
| Anti- GAPDH | Cell Signaling | Cat# 97166S |
| Cycloheximide | Sigma-Aldrich | Cat# C4859-1ML |
| TnT SP6 High-Yield Wheat Germ Protein | Promega | Cat# L3260 |
| Expression System | | |
| Quick-RNA MiniPrep | Zymo | Cat# R1054 |
| GeneJET Genomic DNA Purification Kit | Thermo-Fisher | Cat# K0721 |
| TruSeq RNA Library Prep Kit | Illumina | Cat# RS-122-2001 |
| Alt-R Genome Editing Detection Kit | IDT | Cat# 1075931 |
| SuperScript IV | Thermo-Fisher | Cat# 18091050 |
| Novex 4-12% Tris-Glycine Mini Gels | Thermo-Fisher | Cat# XP04122BOX |
| Novex TBE Gels, 8% | Thermo-Fisher | Cat# EC6215BOX |
| Gibson Assembly Master Mix | NEB | Cat# E2611S |
| Quick Ligation Kit | NEB | Cat# M2200S |
| NotI-HF | NEB | Cat# R3189L |
| AscI | NEB | Cat# R0558L |

### Example 9: CH933 and CH937 Compared to Previously Known ASOs

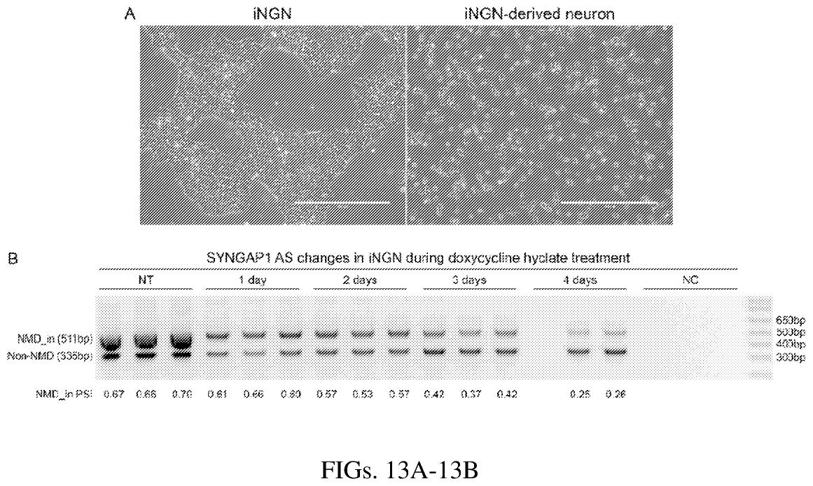

CH933 and CH937 show an improvement over previously known ASOs by at least having a higher efficiency in repressing SYNGAP1 A3SS-NMD and increasing productive transcripts, having lower toxicity (including in neurons), and having a higher validation. SYNGAP1 primarily functions in neurons. Therefore, neurons are an appropriate model to validate the SSO's effect on splice-switching of SYNGAP1. The inventors utilized human induced pluripotent stem cell (hiPSC)-derived neurons as a validation platform. iNGN are hiPSC that can be induced to be neurons by doxycycline treatment for 4 days (FIG. 13A). The inventors showed an increase on the productive transcript of SYNGAP1 during neuronal induction also demonstrated the neuron characteristic of this model (FIG. 13B). Using the previously known SSO ASO71 as a comparison, the inventors assessed the SSOs CH933 and CH937 in the neuron model. The results showed that CH937 has a significantly higher efficiency than ASO71 (FIGS. 6C-6E).

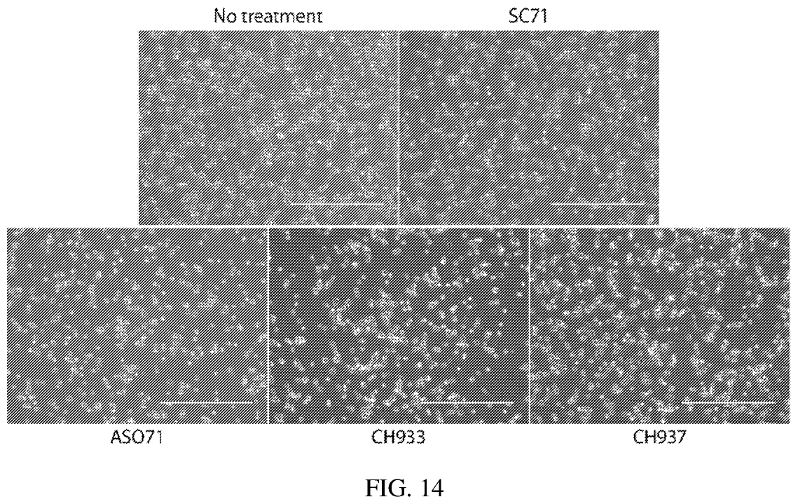

48 hours after ASOs treatment, the inventors observed the cell density and neuronal morphology of neurons. The results demonstrated that CH933 and CH937 groups remained higher density and longer axon of neuron than the ASO71 group (FIG. 14), which shows that CH933 and CH937 have lower toxicity to neurons.

Previous studies examined ASO71 in HEK293 and MEF (mouse embryonic fibroblast). However, neither of them is a neuronal cell type so the inventors don't know their effectivity on neurons. The technology was been tested in hiPSC-derived neurons and showed better performance. Further, the inventors tested CH937 in two human iPSC lines, iNGN and 28126, the results of which also demonstrated CH937 has better efficiency than ASO71 (FIGS. 6I-6K, 12B-12D).

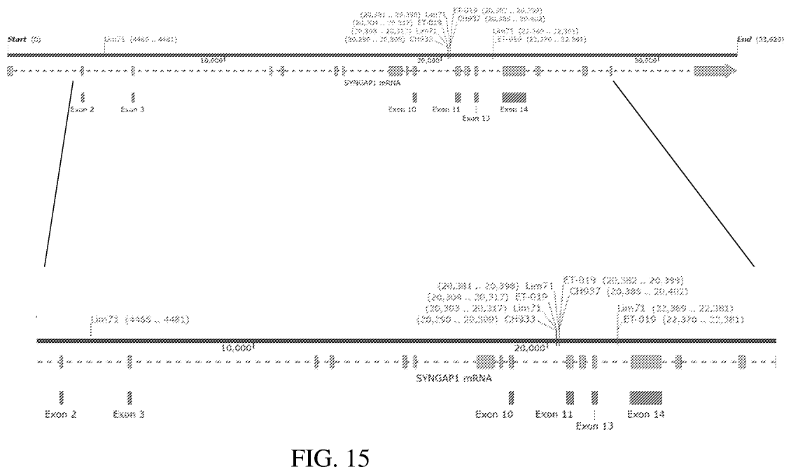

The inventors found the ASOs disclosed herein (including CH933 and CH937) have one binding site in the SYNGAP1 pre-mRNA, compared to the previously known ASOs, which have multiple binding sites (FIG. 15). Having one binding site compared to multiple binding sites can lead to the differences in specificity, efficiency, toxicity, and effectivity shown herein.

### Example 10: Additional ASO Screening and Testing in SH-SY5Y Cells

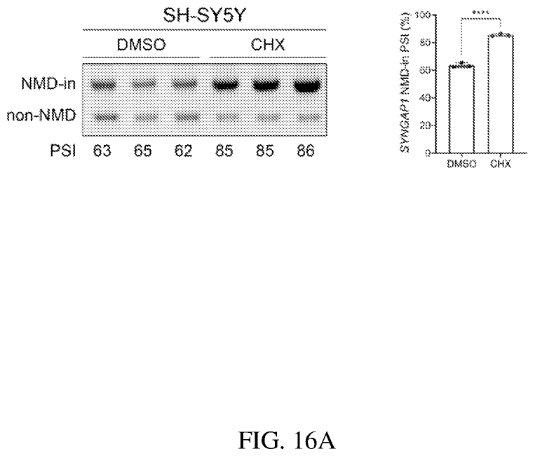

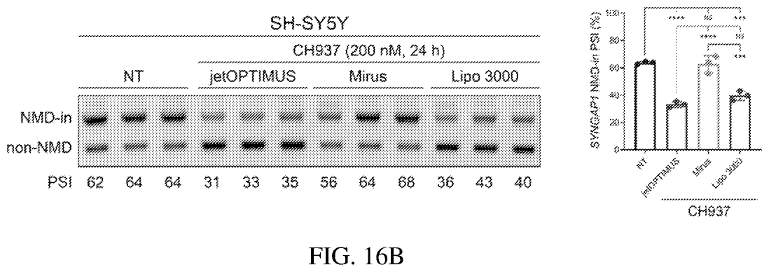

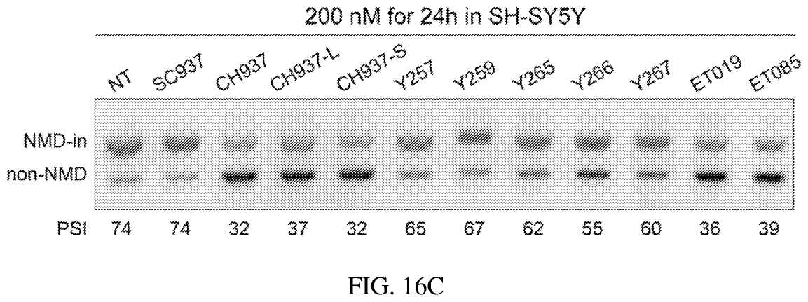

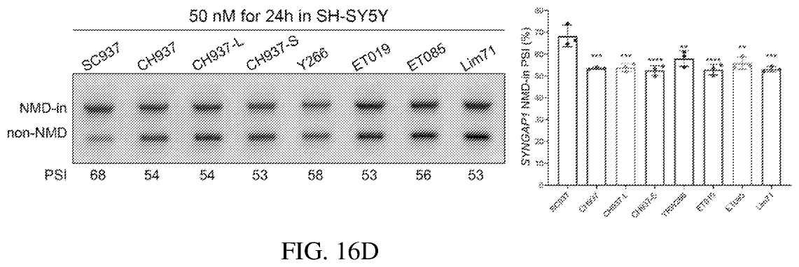

The inventors tested certain ASOs described herein. FIGS. 16A-16D show the effects of these ASOs in a neuroblastoma cell line (SH-SY5Y). Various delivery mechanisms were used, including jetOPTIMUP, Mirus, and Lipofectamine 3000 to deliver CH937 to SH-SY5Y cells. The inventors found jetOPTIMUS performed better than Mirus and Lipofectamine 3000, as measured by percent SYNGAP1 NMD-in PSI (FIG. 16B). These ASOs are also assayed for efficacy and toxicity as disclosed herein. The disclosed herein are administered to cells and/or animal models to determine efficacy and/or toxicity, including at various dose ranges.

### Example 11: Sequences for Certain ASOs Disclosed Herein

Certain ASOs disclosed herein are provided in Table 1.

TABLE 1

| ASO | SEQ ID NO | Sequence |
|---|---|---|
| CH933 | 1 | AGATGGAGGGTCACTTGAGG |
| CH937 | 2 | AGACACGTGGGAGAGAGA |
| CH937-L | 3 | GAGAGACACGTGGGAGAGAGA |
| CH937-S | 4 | GACACGTGGGAGAGAGA |
| YRW266 | 5 | GGAGGGACCGCAGGAGCA |
| YRW265 | 6 | GGGAAGGAGGGACCGCAG |
| YRW267 | 7 | GACCGCAGGAGCAAGATG |
| YRW257 | 8 | GGTAGGAGGTGAGGGTGT |
| YRW259 | 9 | GAGGGTGTGGAAACAGGG |
| Lim71 | 10 | ACGTGGGAGAGAGATGGA |
| ET019 | 11 | CACGTGGGAGAGAGATGG |
| ET085 | 12 | TCCAGGGAACATGCTGAG |

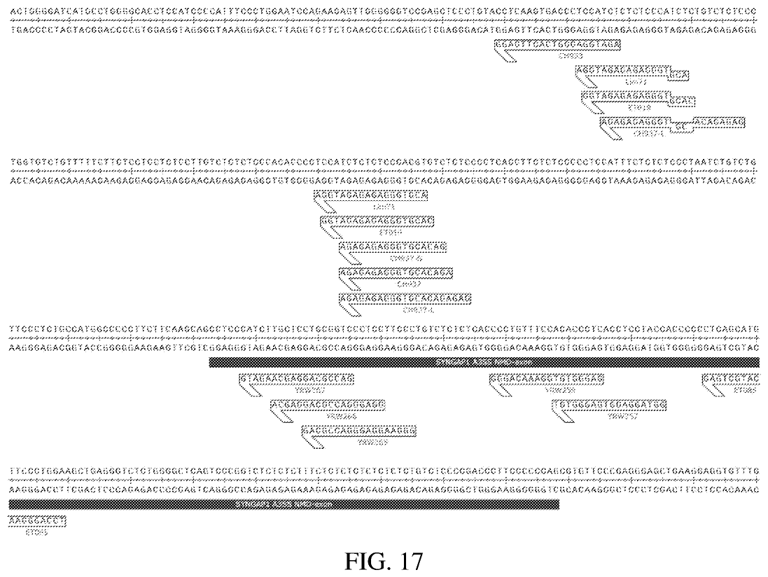

The sequences are shown in FIG. 17 in context of one location within the SYNGAP1 pre-mRNA.

All of the methods disclosed and claimed herein can be made and executed without undue experimentation in light of the present disclosure. While the compositions and methods of this disclosure have been described in terms of preferred aspects, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Araki, Y., Zeng, M., Zhang, M., and Huganir, R. L. (2015). Rapid dispersion of SynGAP from synaptic spines triggers AMPA receptor insertion and spine enlargement during LTP. Neuron 85, 173-189. 10.1016/j.neuron.2014.12.023.
Arias-Cavieres, A., Khuu, M. A., Nwakudu, C. U., Barnard, J. E., Dalgin, G., and Garcia, A. J., 3rd (2020). A HIF1a-Dependent Pro-Oxidant State Disrupts Synaptic Plasticity and Impairs Spatial Memory in Response to Intermittent Hypoxia. eNeuro 7. 10.1523/ENEURO.0024-20.2020.
Berryer, M. H., Chattopadhyaya, B., Xing, P., Riebe, I., Bosoi, C., Sanon, N., Antoine-Bertrand, J., Levesque, M., Avoli, M., Hamdan, F. F., et al. (2016). Decrease of SYNGAP1 in GABAergic cells impairs inhibitory synapse connectivity, synaptic inhibition and cognitive function. Nat Commun 7, 13340. 10.1038/ncomms13340.
Berryer, M. H., Hamdan, F. F., Klitten, L. L., Moller, R. S., Carmant, L., Schwartzentruber, J., Patry, L., Dobrzeniecka, S., Rochefort, D., Neugnot-Cerioli, M., et al. (2013). Mutations in SYNGAP1 cause intellectual disability, autism, and a specific form of epilepsy by inducing haploinsufficiency. Hum Mutat 34, 385-394. 10.1002/humu.22248.
Black, D. L. (2003). Mechanisms of alternative pre-messenger RNA splicing. Annu Rev Biochem 72, 291-336. 10.1146/annurev.biochem.72.121801.161720.
Boutz, P. L., Stoilov, P., Li, Q., Lin, C. H., Chawla, G., Ostrow, K., Shiue, L., Ares, M., Jr., and Black, D. L. (2007). A post-transcriptional regulatory switch in polypyrimidine tract-binding proteins reprograms alternative splicing in developing neurons. Genes Dev 21, 1636-1652. 10.1101/gad.1558107.
Busskamp, V., Lewis, N. E., Guye, P., Ng, A. H., Shipman, S. L., Byrne, S. M., Sanjana, N. E., Mum, J., Li, Y., Li, S., et al. (2014). Rapid neurogenesis through transcriptional activation in human stem cells. Mol Syst Biol 10, 760.10.15252/msb.20145508.
Camp, J. G., Badsha, F., Florio, M., Kanton, S., Gerber, T., Wilsch-Brauninger, M., Lewitus, E., Sykes, A., Hevers, W., Lancaster, M., et al. (2015). Human cerebral organoids recapitulate gene expression programs of fetal neocortex development. Proc Natl Acad Sci USA 112, 15672-15677. 10.1073/pnas.1520760112.
Carvill, G. L., Engel, K. L., Ramamurthy, A., Cochran, J. N., Roovers, J., Stamberger, H., Lim, N., Schneider, A. L., Hollingsworth, G., Holder, D. H., et al. (2018). Aberrant Inclusion of a Poison Exon Causes Dravet Syndrome and Related SCN1A-Associated Genetic Epilepsies. Am J Hum Genet 103, 1022-1029. 10.1016/j.ajhg.2018.10.023.
Chen, H. J., Rojas-Soto, M., Oguni, A., and Kennedy, M. B. (1998). A synaptic Ras-GTPase activating protein (p135 SynGAP) inhibited by CaM kinase II. Neuron 20, 895-904.
Clement, J.P., Aceti, M., Creson, T. K., Ozkan, E. D., Shi, Y., Reish, N.J., Almonte, A. G., Miller, B. H., Wiltgen, B. J., Miller, C. A., et al. (2012). Pathogenic SYNGAP1 mutations impair cognitive development by disrupting maturation of dendritic spine synapses. Cell 151, 709-723. 10.1016/j.cell.2012.08.045.
Creson, T. K., Rojas, C., Hwaun, E., Vaissiere, T., Kilinc, M., Jimenez-Gomez, A., Holder, J. L., Jr., Tang, J., Colgin, L. L., Miller, C. A., and Rumbaugh, G. (2019). Re-expression of SynGAP protein in adulthood improves translatable measures of brain function and behavior. Elife 8. 10.7554/eLife.46752.
Darnell, R. B. (2013). RNA protein interaction in neurons. Annu Rev Neurosci 36, 243-270. 10.1146/annurev-neuro-062912-114322.
Finkel, R. S., Mercuri, E., Darras, B. T., Connolly, A. M., Kuntz, N. L., Kirschner, J., Chiriboga, C. A., Saito, K., Servais, L., Tizzano, E., et al. (2017). Nusinersen versus Sham Control in Infantile-Onset Spinal Muscular Atrophy. N Engl J Med 377, 1723-1732. 10.1056/NEJMoa1702752.
Gill, D. F., and Hansel, C. (2020). Muscarinic Modulation of SK2-Type K(+) Channels Promotes Intrinsic Plasticity in L2/3 Pyramidal Neurons of the Mouse Primary Somatosensory Cortex. eNeuro 7. 10.1523/ENEURO.0453-19.2020.
Hamdan, F. F., Gauthier, J., Spiegelman, D., Noreau, A., Yang, Y., Pellerin, S., Dobrzeniecka, S., Cote, M., Perreau-Linck, E., Carmant, L., et al. (2009). Mutations in SYNGAP1 in autosomal nonsyndromic mental retardation. N Engl J Med 360, 599-605. 10.1056/NEJMoa0805392.
Han, Z., Chen, C., Christiansen, A., Ji, S., Lin, Q., Anumonwo, C., Liu, C., Leiser, S. C., Meena, Aznarez, I., et al. (2020). Antisense oligonucleotides increase Scn1a expression and reduce seizures and SUDEP incidence in a mouse model of Dravet syndrome. Sci Transl Med 12. 10.1126/scitranslmed.aaz6100.
Hua, Y., Sahashi, K., Rigo, F., Hung, G., Horev, G., Bennett, C. F., and Krainer, A. R. (2011). Peripheral SMN restoration is essential for long-term rescue of a severe spinal muscular atrophy mouse model. Nature 478, 123-126.10.1038/nature10485.
Kim, J., Hu, C., Moufawad El Achkar, C., Black, L. E., Douville, J., Larson, A., Pendergast, M. K., Goldkind, S. F., Lee, E. A., Kuniholm, A., et al. (2019). Patient-Customized Oligonucleotide Therapy for a Rare Genetic Disease. N Engl J Med 381, 1644-1652. 10.1056/NEJMoa1813279.
Kim, J. H., Lee, H. K., Takamiya, K., and Huganir, R. L. (2003). The role of synaptic GTPase-activating protein in neuronal development and synaptic plasticity. J Neurosci 23, 1119-1124.
Kim, J. H., Liao, D., Lau, L. F., and Huganir, R. L. (1998). SynGAP: a synaptic RasGAP that associates with the PSD-95/SAP90 protein family. Neuron 20, 683-691.
Kim, M. J., Dunah, A. W., Wang, Y. T., and Sheng, M. (2005). Differential roles of NR2A- and NR2B-containing NMDA receptors in Ras-ERK signaling and AMPA receptor trafficking. Neuron 46, 745-760. 10.1016/j.neuron.2005.04.031. Kole, R., Krainer, A. R., and Altman, S. (2012). RNA therapeutics: beyond RNA interference and antisense oligonucleotides. Nat Rev Drug Discov 11, 125-140. 10.1038/nrd3625.
Komiyama, N. H., Watabe, A. M., Carlisle, H. J., Porter, K., Charlesworth, P., Monti, J., Strathdee, D. J., O'Carroll, C. M., Martin, S. J., Morris, R. G., et al. (2002). SynGAP regulates ERK/MAPK signaling, synaptic plasticity, and learning in the complex with postsynaptic density 95 and NMDA receptor. J Neurosci 22, 9721-9732.
Lareau, L. F., Inada, M., Green, R. E., Wengrod, J. C., and Brenner, S. E. (2007). Unproductive splicing of SR genes associated with highly conserved and ultraconserved DNA elements. Nature 446, 926-929. 10.1038/nature05676.
Li, Y. I., Sanchez-Pulido, L., Haerty, W., and Ponting, C.P. (2015). RBFOX and PTBP1 proteins regulate the alternative splicing of micro-exons in human brain transcripts. Genome Res 25, 1-13. 10.1101/gr.181990.114.
Licatalosi, D. D., Yano, M., Fak, J. J., Mele, A., Grabinski, S. E., Zhang, C., and Darnell, R. B. (2012). Ptbp2 represses adult-specific splicing to regulate the generation of neuronal precursors in the embryonic brain. Genes Dev 26, 1626-1642. 10.1101/gad.191338.112.
Lim, K. H., Han, Z., Jeon, H. Y., Kach, J., Jing, E., Weyn-Vanhentenryck, S., Downs, M., Corrionero, A., Oh, R., Scharner, J., et al. (2020). Antisense oligonucleotide modulation of non-productive alternative splicing upregulates gene expression. Nat Commun 11, 3501. 10.1038/s41467-020-17093-9.
Lin, L., Zhang, M., Stoilov, P., Chen, L., and Zheng, S. (2020). Developmental Attenuation of Neuronal Apoptosis by Neural-Specific Splicing of Bakl Microexon. Neuron 107, 1180-1196 e1188. 10.1016/j.neuron.2020.06.036.
Linares, A. J., Lin, C. H., Damianov, A., Adams, K. L., Novitch, B. G., and Black, D. L. (2015). The splicing regulator PTBP1 controls the activity of the transcription factor Pbx1 during neuronal differentiation. Elife 4, e09268.
10.7554/eLife.09268.
Lindsay, S. J., Xu, Y., Lisgo, S. N., Harkin, L. F., Copp, A. J., Gerrelli, D., Clowry, G. J., Talbot, A., Keogh, M. J., Coxhead, J., et al. (2016). HDBR Expression: A Unique Resource for Global and Individual Gene Expression Studies during Early Human Brain Development. Front Neuroanat 10, 86. 10.3389/fnana.2016.00086.
Michaelson, S. D., Ozkan, E. D., Aceti, M., Maity, S., Llamosas, N., Weldon, M., Mizrachi, E., Vaissiere, T., Gaffield, M. A., Christie, J. M., et al. (2018). SYNGAP1 heterozygosity disrupts sensory processing by reducing touch-related activity within somatosensory cortex circuits. Nat Neurosci 21, 1-13. 10.1038/s41593-018-0268-0.
Ozkan, E. D., Creson, T. K., Kramar, E. A., Rojas, C., Seese, R. R., Babyan, A. H., Shi, Y., Lucero, R., Xu, X., Noebels, J. L., et al. (2014). Reduced cognition in Syngap1 mutants is caused by isolated damage within developing forebrain excitatory neurons. Neuron 82, 1317-1333. 10.1016/j.neuron.2014.05.015.
Pitts, M. W. (2018). Barnes Maze Procedure for Spatial Learning and Memory in Mice. Bio Protoc 8. 10.21769/bioprotoc.2744.
Prchalova, D., Havlovicova, M., Sterbova, K., Stranecky, V., Hancarova, M., and Sedlacek, Z. (2017). Analysis of 31-year-old patient with SYNGAP1 gene defect points to importance of variants in broader splice regions and reveals developmental trajectory of SYNGAP1-associated phenotype: case report. BMC Med Genet 18, 62. 10.1186/s12881-017-0425-4.
Raj, B., and Blencowe, B. J. (2015). Alternative Splicing in the Mammalian Nervous System: Recent Insights into Mechanisms and Functional Roles. Neuron 87, 14-27. 10.1016/j.neuron.2015.05.004.
Rodriguez-Palmero, A., Boerrigter, M. M., Gomez-Andres, D., Aldinger, K. A., Marcos-Alcalde, I., Popp, B., Everman, D. B., Lovgren, A. K., Arpin, S., Bahrambeigi, V., et al. (2021). DLG4-related synaptopathy: a new rare brain disorder. Genet Med 23, 888-899. 10.1038/s41436-020-01075-9.
Rumbaugh, G., Adams, J.P., Kim, J. H., and Huganir, R. L. (2006). SynGAP regulates synaptic strength and mitogen-activated protein kinases in cultured neurons. Proc Natl Acad Sci USA 103, 4344-4351. 10.1073/pnas.0600084103.
Satterstrom, F. K., Kosmicki, J. A., Wang, J., Breen, M. S., De Rubeis, S., An, J. Y., Peng, M., Collins, R., Grove, J., Klei, L., et al. (2020). Large-Scale Exome Sequencing Study Implicates Both Developmental and Functional Changes in the Neurobiology of Autism. Cell 180, 568-584 e523. 10.1016/j.cell.2019.12.036.
Sauliere, J., Sureau, A., Expert-Bezancon, A., and Marie, J. (2006). The polypyrimidine tract binding protein (PTB) represses splicing of exon6B from the beta-tropomyosin pre-mRNA by directly interfering with the binding of the U2AF65 subunit. Mol Cell Biol 26, 8755-8769. 10.1128/MCB.00893-06.
Sudhof, T. C. (2018). Towards an Understanding of Synapse Formation. Neuron 100, 276-293. 10.1016/j.neuron.2018.09.040.
Vlaskamp, D. R. M., Shaw, B. J., Burgess, R., Mei, D., Montomoli, M., Xie, H., Myers, C. T., Bennett, M. F., XiangWei, W., Williams, D., et al. (2019). SYNGAP1 encephalopathy: A distinctive generalized developmental and epileptic encephalopathy. Neurology 92, e96-e107. 10.1212/WNL.0000000000006729.
Vuong, C. K., Black, D. L., and Zheng, S. (2016). The neurogenetics of alternative splicing. Nat Rev Neurosci 17, 265-281. 10.1038/nrn.2016.27.
Weyn-Vanhentenryck, S. M., Mele, A., Yan, Q., Sun, S., Farny, N., Zhang, Z., Xue, C., Herre, M., Silver, P. A., Zhang, M. Q., et al. (2014). HITS-CLIP and integrative modeling define the Rbfox splicing-regulatory network linked to brain development and autism. Cell Rep 6, 1139-1152. 10.1016/j.celrep.2014.02.005.
Yan, Q., Weyn-Vanhentenryck, S. M., Wu, J., Sloan, S. A., Zhang, Y., Chen, K., Wu, J. Q., Barres, B. A., and Zhang, C. (2015). Systematic discovery of regulated and conserved alternative exons in the mammalian brain reveals NMD modulating chromatin regulators. Proc Natl Acad Sci USA 112, 3445-3450. 10.1073/pnas.1502849112.
Yoon, S. J., Elahi, L. S., Pasca, A. M., Marton, R. M., Gordon, A., Revah, O., Miura, Y., Walczak, E. M., Holdgate, G. M., Fan, H. C., et al. (2019). Reliability of human cortical organoid generation. Nat Methods 16, 75-78. 10.1038/s41592-018-0255-0.
Zeng, M., Shang, Y., Araki, Y., Guo, T., Huganir, R. L., and Zhang, M. (2016). Phase Transition in Postsynaptic Densities Underlies Formation of Synaptic Complexes and Synaptic Plasticity. Cell 166, 1163-1175 e1112.
10.1016/j.cell.2016.07.008.
Zhang, X., Chen, M. H., Wu, X., Kodani, A., Fan, J., Doan, R., Ozawa, M., Ma, J., Yoshida, N., Reiter, J. F., et al. (2016). Cell-Type-Specific Alternative Splicing Governs Cell Fate in the Developing Cerebral Cortex. Cell 166, 1147-1162 e1115.
10.1016/j.cell.2016.07.025.
Zheng, S., Gray, E. E., Chawla, G., Porse, B. T., O'Dell, T. J., and Black, D. L. (2012). PSD-95 is post-transcriptionally repressed during early neural development by PTBP1 and PTBP2. Nat Neurosci 15, 381-388, S381. 10.1038/nn.3026.
Zoghbi, H. Y., and Bear, M. F. (2012). Synaptic dysfunction in neurodevelopmental disorders associated with autism and intellectual disabilities. Cold Spring Harb Perspect Biol 4. 10.1101/cshperspect.a009886.

What is claimed is:

1. A method of increasing a protein produced from a SYNGAP1 pre-mRNA in a cell, the method comprising contacting the cell with at least one antisense oligonucleotide (ASO), wherein at least one of the ASOs are fully complementary to only one contiguous region of the SYNGAP1 pre-mRNA in the cell.

2. The method of claim 1, wherein the cell comprises a neuron.

3. The method of claim 1 or 2, wherein the cell comprises a haploinsufficiency of the SYNGAP1 gene.

4. The method of any one of claims 1-3, wherein the cell comprises a loss-of-function mutation in the SYNGAP1 gene.

5. The method of any one of claims 1-4, wherein the one contiguous region of the SYNGAP1 pre-mRNA is an intronic region of the SYNGAP1 gene.

6. The method of any one of claims 1-5, wherein the one contiguous region of the SYNGAP1 pre-mRNA is in intron 10 of the SYNGAP1 gene.

7. The method of any one of claims 1-6, wherein the one contiguous region of the SYNGAP1 pre-mRNA is a polypyrimidine tract binding protein (PTBP)-binding motif in the SYNGAP1 gene.

8. The method of any one of claims 1-7, wherein at least one ASO has at least 90%, 95%, or 100% sequence identity to SEQ ID NO:1.

9. The method of any one of claims 1-8, wherein at least one ASO has at least 90%, 95%, or 100% sequence identity to SEQ ID NO:2.

10. The method of any one of claims 1-9, wherein at least one ASO has at least 90%, 95%, or 100% sequence identity to SEQ ID NO:3.

11. The method of any one of claims 1-10, wherein at least one ASO has at least 90%, 95%, or 100% sequence identity to SEQ ID NO:4.

12. The method of any one of claims 1-11, wherein at least one ASO has at least 90%, 95%, or 100% sequence identity to SEQ ID NO:5.

13. The method of any one of claims 1-12, wherein the ASO consists of an ASO with at least 90%, 95%, or 100% sequence identity to SEQ ID NO:1, SEQ ID NO:2, SEQ ID NO:3, SEQ ID NO:4, or SEQ ID NO:5.

14. The method of any one of claims 1-13, wherein the ASOs comprise two or more ASOs having at least 90%, 95%, or 100% sequence identity to SEQ ID NO:1, SEQ ID NO:2, SEQ ID NO:3, SEQ ID NO:4, or SEQ ID NO:5.

15. The method of any one of claims 1-14, wherein the ASO is between 17-21 nucleotides in length.

16. The method of any one of claims 1-14, wherein the ASO is 18 nucleotides in length.

17. The method of any one of claims 1-14, wherein the ASO is 20 nucleotides in length.

18. The method of any one of claims 1-17, wherein the cell is contacted with an amount of the ASO sufficient to reduce nonsense-mediated decay of mRNA derived from the SYNGAP1 pre-mRNA by greater than 30%.

19. The method of any one of claims 1-18, wherein the cell is contacted with an amount of the ASO sufficient to increase the protein produced from the SYNGAP1 pre-mRNA to an amount that is more than 1.5-fold greater than an amount of protein produced from the SYNGAP1 pre-mRNA present in a cell not contacted with the ASO.

20. A method of treating a neurological disorder in a patient, the method comprising administering to the patient a therapeutically effective amount of at least one antisense oligonucleotide (ASO), and wherein the ASO is complementary to one contiguous region of a SYNGAP1 pre-mRNA.

21. The method of claim 20, wherein a neuron in the patient has at least one loss of function mutation in a SYNGAP1 gene.

22. The method of claim 21, wherein the loss of function mutation in the SYNGAP1 gene causes nonsense-mediated decay of mRNA transcribed from the SYNGAP1 gene absent the ASO binding.

23. The method of any one of claims 20-22, wherein the neuron has a haploinsufficiency of the SYNGAP1 gene.

24. The method of any one of claims 20-23, wherein the neurological disorder is autism, an intellectual disability, and/or epilepsy.

25. The method of any one of claims 20-24, wherein the one contiguous region of the SYNGAP1 pre-mRNA is an intronic region of the SYNGAP1 gene.

26. The method of any one of claims 20-25, wherein the one contiguous region of the SYNGAP1 pre-mRNA is in intron 10 of the SYNGAP1 gene.

27. The method of any one of claims 20-26, wherein the one contiguous region of the SYNGAP1 pre-mRNA is a polypyrimidine tract binding protein (PTBP)-binding motif in the SYNGAP1 gene.

28. The method of any one of claims 20-27, wherein at least one ASO has at least 90%, 95%, or 100% sequence identity to SEQ ID NO:1.

29. The method of any one of claims 20-28, wherein at least one ASO has at least 90%, 95%, or 100% sequence identity to SEQ ID NO:2.

30. The method of any one of claims 20-29, wherein at least one ASO has at least 90%, 95%, or 100% sequence identity to SEQ ID NO:3.

31. The method of any one of claims 20-30, wherein at least one ASO has at least 90%, 95%, or 100% sequence identity to SEQ ID NO:4.

32. The method of any one of claims 20-31, wherein at least one ASO has at least 90%, 95%, or 100% sequence identity to SEQ ID NO:5.

33. The method of any one of claims 20-32, wherein the ASO consists of an ASO with at least 90%, 95%, or 100% sequence identity to SEQ ID NO:1, SEQ ID NO:2, SEQ ID NO:3, SEQ ID NO:4, or SEQ ID NO:5.

34. The method of any one of claims 1-33, wherein the ASOs comprise two or more ASOs having at least 90%, 95%, or 100% sequence identity to SEQ ID NO:1, SEQ ID NO:2, SEQ ID NO:3, SEQ ID NO:4, or SEQ ID NO:5.

35. An antisense oligonucleotide (ASO) comprising a sequence complementary to one contiguous region of a SYNGAP1 pre-mRNA, wherein the ASO is capable of binding to the contiguous region of the SYNGAP1 pre-mRNA in a manner that blocks an alternative splicing of the SYNGAP1 pre-mRNA that leads to nonsense-mediated decay of an mRNA derived from the SYNGAP1 pre-mRNA.

36. The ASO of claim 35, wherein the one contiguous region of the SYNGAP1 pre-mRNA is an intronic region of the SYNGAP1 gene.

37. The ASO of claim 35 or 36, wherein the one contiguous region of the SYNGAP1 pre-mRNA is in intron 10 of the SYNGAP1 gene.

38. The ASO of any one of claims 35-37, wherein the one contiguous region of the SYNGAP1 pre-mRNA is a polypyrimidine tract binding protein (PTBP)-binding motif in the SYNGAP1 gene.

39. The ASO of claim 35, wherein the one contiguous region of the SYNGAP1 pre-mRNA is an exonic region of the SYNGAP1 gene.

40. The ASO of claim 39, wherein the exonic region is in exon11 of the SYNGAP1 pre-mRNA.

41. The ASO of any one of claims 35-40, wherein the ASO is isolated.

42. An antisense oligonucleotide having 90%, 95%, or 100% sequence identity to the sequence of SEQ ID NO:1, wherein the antisense oligonucleotide is isolated.

43. An antisense oligonucleotide having 90%, 95%, or 100% sequence identity to the sequence of SEQ ID NO:2, wherein the antisense oligonucleotide is isolated.

44. An antisense oligonucleotide having 90%, 95%, or 100% sequence identity to the sequence of SEQ ID NO:3, wherein the antisense oligonucleotide is isolated.

45. An antisense oligonucleotide having 90%, 95%, or 100% sequence identity to the sequence of SEQ ID NO:4, wherein the antisense oligonucleotide is isolated.

46. An antisense oligonucleotide having 90%, 95%, or 100% sequence identity to the sequence of SEQ ID NO:5, wherein the antisense oligonucleotide is isolated.

47. An isolated antisense oligonucleotide having 90%, 95%, or 100% sequence identity to the sequence of SEQ ID NO:1.

48. An isolated antisense oligonucleotide having 90%, 95%, or 100% sequence identity to the sequence of SEQ ID NO:2.

49. An isolated antisense oligonucleotide having 90%, 95%, or 100% sequence identity to the sequence of SEQ ID NO:3.

50. An isolated antisense oligonucleotide having 90%, 95%, or 100% sequence identity to the sequence of SEQ ID NO:4.

51. An isolated antisense oligonucleotide having 90%, 95%, or 100% sequence identity to the sequence of SEQ ID NO:5.

52. A pharmaceutical composition comprising the ASO of any one of claims 35-51.

53. A method of treating a neurological disorder in a patient comprising administering a therapeutically effective amount of the pharmaceutical composition of claim 52 to the patient.


SEQUENCE LISTING (12 sequences)

SEQ ID NO: 1 - DNA, 20 bp, synthetic construct
  source 1..20: mol_type = other DNA
agatggagggtcacttgagg

SEQ ID NO: 2 - DNA, 18 bp, synthetic construct
  source 1..18: mol_type = other DNA
agacacgtgggagagaga

SEQ ID NO: 3 - DNA, 21 bp, synthetic construct
  source 1..21: mol_type = other DNA
gagagacacgtgggagagaga

SEQ ID NO: 4 - DNA, 17 bp, synthetic construct
  source 1..17: mol_type = other DNA
gacacgtgggagagaga

SEQ ID NO: 5 - DNA, 18 bp, synthetic construct
  source 1..18: mol_type = other DNA
ggagggaccgcaggagca

SEQ ID NO: 6 - DNA, 18 bp, synthetic construct
  source 1..18: mol_type = other DNA
gggaaggagggaccgcag

SEQ ID NO: 7 - DNA, 18 bp, synthetic construct
  source 1..18: mol_type = other DNA
gaccgcaggagcaagatg

SEQ ID NO: 8 - DNA, 18 bp, synthetic construct
  source 1..18: mol_type = other DNA
ggtaggaggtgagggtgt

SEQ ID NO: 9 - DNA, 18 bp, synthetic construct
  source 1..18: mol_type = other DNA
gagggtgtggaaacaggg

SEQ ID NO: 10 - DNA, 18 bp, synthetic construct
  source 1..18: mol_type = other DNA
acgtgggagagagatgga

SEQ ID NO: 11 - DNA, 18 bp, synthetic construct
  source 1..18: mol_type = other DNA
cacgtgggagagagatgg

SEQ ID NO: 12 - DNA, 18 bp, synthetic construct
  source 1..18: mol_type = other DNA
tccagggaacatgctgag